(19) United States
(12) Patent Application Publication
(10) Pub. No.: US 2025/0027021 A1
(43) Pub. Date: Jan. 23, 2025

(54) CELL CULTURE SAMPLING FROM FIXED BED BIOREACTOR METHODS AND APPARATUS

(71) Applicant: CORNING INCORPORATED, CORNING, NY (US)

(72) Inventors: Tristan Yale Brawn, Billerica, MA (US); Michael Francis Finnegan, Los Altos, CA (US); Jacob Dylan Freake, Cambridge, MA (US); Daniel Thomas Manning, Groton, MA (US); Steven Eric Meyer, Arlington, MA (US); Douglas Grosvenor Sabin, Marblehead, MA (US); Joel Andrew Schultes, Corning, NY (US)

(21) Appl. No.: 18/714,892

(22) PCT Filed: Nov. 21, 2022
(86) PCT No.: PCT/US2022/050563

§ 371 Date: May 30, 2024

Related U.S. Application Data
(60) Provisional application No. 63/284,371, filed on Nov. 30, 2021.
(60) Provisional application No. 63/284,256, filed on Nov. 30, 2021.
(60) Provisional application No. 63/284,169, filed on Nov. 30, 2021.

(51) Int. Cl. C12M 1/12; C12M 1/00

(52) U.S. Cl. CPC C12M 25/18; C12M 23/04; C12M 23/34; C12M 23/38

(57) ABSTRACT

A fixed bed bioreactor assembly for culturing cells is provided and methods for sampling cell culture substrates from such assemblies. The assembly includes a bioreactor vessel having an interior space for culturing cells and a sidewall at least partly defining the interior space; and a plurality of cell culture substrate layers in the interior space, each layer having a structurally defined surface for culturing cells thereon. The structurally defined surface defines an ordered and regular array of openings through a thickness of the layer. The assembly further includes a sleeve at least partially surrounding the plurality of cell culture substrate layers and having at least one sample access window. The sample access window includes an opening in the sleeve to allow one or more layers of the cell culture substrate to be removed from the sleeve through the opening.

This application claims the benefit of priority under 35 U.S.C. § 119 of U.S. Provisional Application Ser. No. 63/284,371 filed on Nov. 30, 2021, U.S. Provisional Application Ser. No. 63/284,256 filed on Nov. 30, 2021, and U.S. Provisional Application Ser. No. 63/284, 169 filed on Nov. 30, 2021, the contents of which are relied upon and incorporated herein by reference in their entirety.

### FIELD OF THE DISCLOSURE

This disclosure generally relates to substrates for culturing cells, and bioreactors for housing the same, that enable sampling of the substrate during cell culture. In particular, the present disclosure relates to cell culturing substrates and bioreactors incorporating such substrates that allow for removal of samples from the bioreactor, to allow for aseptic sampling of portions of the substrate during and/or after the cell culture process to monitor the health and progress of the culture and other processes.

### BACKGROUND

In the bioprocessing industry, large-scale cultivation of cells is performed for purposes of the production of hormones, enzymes, antibodies, vaccines, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success.

A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and HYPERStack® vessels. These approaches can have significant drawbacks, including the difficulty in achieving cellular density high enough to make it feasible for large scale production of therapies or cells.

Alternative methods have been suggested to increase volumetric density of cultured cells. These include microcarrier cultures performed in stir tanks. In this approach, cells that are attached to the surface of microcarriers are subject to constant shear stress, resulting in a significant impact on proliferation and culture performance. Another example of a high-density cell culture system is a hollow fiber bioreactor, in which cells may form large three-dimensional aggregates as they proliferate in the interspatial fiber space. However, the cells growth and performance are significantly inhibited by the lack nutrients. To mitigate this problem, these bioreactors are made small and are not suitable for large scale manufacturing

Another example of a high-density culture system for anchorage dependent cells is a packed-bed bioreactor system. In this this type of bioreactor, a cell substrate is used to provide a surface for the attachment of adherent cells. Medium is perfused along the surface or through the semi-porous substrate to provide nutrients and oxygen needed for the cell growth. For example, packed bed bioreactor systems that contain a packed bed of support or matrix systems to entrap the cells have been previously disclosed U.S. Pat. Nos. 4,833,083; 5,501,971; and 5,510,262. Packed bed matrices usually are made of porous particles as substrates or non-woven microfibers of polymer. Such bioreactors function as recirculation flow-through bioreactors. One of the significant issues with such bioreactors is the non-uniformity of cell distribution inside the packed bed. For example, the packed bed functions as depth filter with cells predominantly trapped at the inlet regions, resulting in a gradient of cell distribution during the inoculation step. In addition, due to random fiber packaging, flow resistance and cell trapping efficiency of cross sections of the packed bed are not uniform. For example, medium flows fast though the regions with low cell packing density and flows slowly through the regions where resistance is higher due to higher number of entrapped cells. This creates a channeling effect where nutrients and oxygen are delivered more efficiently to regions with lower volumetric cells densities and regions with higher cell densities are being maintained in suboptimal culture conditions.

Another significant drawback of packed bed systems disclosed in a prior art is the inability to efficiently harvest intact viable cells at the end of culture process. Harvesting of cells is important if the end product is cells, or if the bioreactor is being used as part of a “seed train,” where a cell population is grown in one vessel and then transferred to another vessel for further population growth. U.S. Pat. No. 9,273,278 discloses a bioreactor design to improve the efficiency of cell recovery from the packed bed during cells harvesting step. It is based on loosening the packed bed matrix and agitation or stirring of packed bed particles to allow porous matrices to collide and thus detach the cells. However, this approach is laborious and may cause significant cells damage, thus reducing overall cell viability.

Some existing bioreactor solutions use small strips of cell substrate material consisting of randomly oriented fibers in a non-woven arrangement. These strips are packed into a vessel to create a packed bed. However, as with similar solutions on the market, there are drawbacks to this type of packed-bed substrate. Specifically, non-uniform packing of the substrate strips creates visible channels within the packed bed, leading to preferential and non-uniform media flow and nutrient distribution through the packed bed. Studies of such systems have noted a “systemic inhomogeneous distribution of cells, with their number increasing from top to bottom of fixed bed,” as well as a “nutrient gradient . . . leading to restricted cell growth and production,” all of which lead to the “unequal distribution of cells [that] may impair transfection efficiency.” (Rational plasmid design and bioprocess optimization to enhance recombinant adeno-associated virus (AAV) productivity in mammalian cells. Biotechnol. J. 2016, 11, 290-297). Studies have noted that agitation of the packed bed may improve dispersion, but would have other drawbacks (i.e., “necessary agitation for better dispersion during inoculation and transfection would induce increased shear stress, in turn leading to reduced cell viability.” Id.). Another study noted that the uneven distribution of cells makes monitoring of the cell population using biomass sensors difficult (“ . . . if the cells are unevenly distributed, the biomass signal from the cells on the top carriers may not show the general view of the entire bioreactor.” Process Development of Adenoviral Vector Production in Fixed Bed Bioreactor: From Bench to Commercial Scale. Human Gene Therapy, Vol. 26, No. 8, 2015).

In addition, because of the random arrangement of fibers in the substrate strips and the variation in packing of strips between one packed bed and another in bioreactors like those discussed above, it can be difficult for customers to predict cell culture performance, since the substrate varies between cultures. Furthermore, the randomly packed substrate, which themselves have random structure, makes efficiently harvesting cells very difficult or impossible, as it is believed that cells are entrapped by the packed bed.

While manufacturing of viral vectors for early-phase clinical trials is possible with existing platforms, there is a need for a platform that can produce high-quality product in greater numbers in order to reach late-stage commercial manufacturing scale.

In addition, it is desirable to be able to monitor bioreactors used to culture cells or make AAV or to create a seed train to facilitate cell expansion for biochemical production. When using adherent cell reactors, samples of the growth media do not contain cells, or at least not to an extent that is useful for monitoring the state of the culture on the adherent substrate.

There is a need for cell culture matrices, systems, and methods that enable culturing of cells in a high-density format, with uniform cell distribution, and easily attainable and increased harvesting yields, while also enabling users to monitor the state of the cell culture process by examining the cells on the substrate during and/or after the cell culture process, including monitoring the substrate aseptically.

### SUMMARY

According to embodiments of this disclosure, a cell culture substrate is disclosed that allows for sampling all or a portion of the substrate to monitor the status or health of the cell culture. Embodiments include a multilayered fixed bed cell culture matrix with one or more layers specifically designed to enable this sampling. Embodiments also include a fixed bed bioreactor with such cell culture substrates and/or matrices.

To be able to estimate the number of cells and their distribution and health in an adherent cell bioreactor bed, one method uses the removal of a portion of the substrate in the midst of the cell culture or cell expansion process. By sampling during the process, information about the cell culture run can be used to assess the quality and performance of the process. Cell count can be estimated from the sample and growth can be monitored by sampling at different times. This information can be used to develop and optimize performance of specific biological processes such as seed train and viral vector production. In production, runs that are contaminated or out of specification, can be terminated to reduce the cost of running the process to its end without a satisfactory result. Growth media and lost production time represent significant cost for typical biological processes. Embodiments of this disclosure allow all or portions of the fixed bed cell culture substrate to be removed from the housing to give users access to the bed without destroying the bed or bioreactor vessel. This allows any portion or select portions of the fixed bed to be assessed. The bed can also be accessed after the cell culture process or harvesting of the desired component is completed for a “post-mortem” analysis of the cell culture.

According to embodiments of this disclosure, a fixed bed bioreactor assembly for culturing cells and sampling the substrate during cell culture is provided. The fixed bed bioreactor assembly comprises a bioreactor vessel comprising an interior space for culturing cells and a sidewall at least partly defining the interior space; a plurality of cell culture substrate layers disposed in the interior space, each layer having a structurally defined surface for culturing cells thereon, the structurally defined surface defining an ordered and regular array of openings through a thickness of the layer; and a sleeve at least partially surrounding the plurality of cell culture substrate layers and comprising at least one sample access window, the sample access window comprising an opening in the sleeve configured to allow one or more layers of the cell culture substrate to be removed from the sleeve through the opening.

According to embodiments of this disclosure, a method of sampling a cell culture substrate from a bioreactor vessel. The method comprises removing a sleeve of cell culture substrate layers stacked within the sleeve from a bioreactor vessel through a top or bottom of a bioreactor vessel; removing one or more layers of the cell culture substrate through a sample access window disposed in a side of the sleeve, the sample access window being an opening formed in the sleeve and exposed at least a portion of the cell culture substrate layers.

According to embodiments of this disclosure, a fixed bed bioreactor system for culturing cells is provided. The system comprises: a bioreactor vessel comprising an interior space for culturing cells and a sidewall at least partly defining the interior space; at least one port in the sidewall of the bioreactor vessel; a cell culture substrate disposed in the interior space; and a sampling reel disposed on an exterior of the bioreactor vessel, the sampling reel being configured to rotate about its central axis to aseptically extract a sample substrate from interior space.

According to aspects of embodiments, the sampling reel comprises a passthrough opening through which the substrate sample is able to pass from the interior space and into the sampling reel. The sampling reel can be affixed to the exterior of the bioreactor vessel wall via a saddle housing forming at least a portion of the outer shell of the sampling reel. The passthrough opening of the sampling reel is aligned with the at least one port of the bioreactor vessel. The sampling reel further comprises a housing cap forming at least a portion of the outer shell of the sampling reel. The sampling reel further comprises a sample opening, wherein the sample opening is disposed between the housing cap and saddle housing. The sample opening is configured to allow a substrate sample to be extracted from the sampling reel via the sample opening to an exterior of the bioreactor system. The sampling reel further comprises a spool configured to grip a portion of the sample substrate, the spool being rotatable such that when the spool rotates, the sample substrate is wound around the spool. The sampling reel further comprises a sample drum disposed between the spool and the outer shell of the sampling reel. The sampling reel can further include a series of stationary o-rings affixed to the saddle housing, and configured to create seals in a space between the saddle housing and the sample drum, to enable the aseptic sampling of the sample substrate.

According to aspects of embodiments, the sample drum further comprises a drum opening configured to allow the sample substrate to pass within the sample drum to the spool. The sampling reel is configured to rotate in a first direction to extract the sample substrate from the interior space, and to rotate in a second direction opposite to the first direction to release the sample substrate from the sampling reel to an exterior of the bioreactor system. The substrate sample lifted through an opening of the sampling reel to an exterior of the bioreactor system via a curved or wedged surface on an edge of the passthrough opening.

According to embodiments of this disclosure, a fixed bed bioreactor system for culturing cells is provided. The system comprises a bioreactor vessel comprising an interior space for culturing cells and a sidewall at least partly defining the interior space; at least one port in the sidewall of the bioreactor vessel; and a cell culture substrate disposed in the interior space and having a structurally defined surface for culturing cells thereon, the structurally defined surface defining an ordered and regular array of openings through a thickness of the cell culture substrate. At least a portion of the cell culture substrate comprises a sample substrate, the sample substrate being defined by a separation boundary between the sample substrate and a remainder of the cell culture substrate. The separation boundary is configured to separate the sample substrate from the remainder of the cell culture substrate, and the at least one port is sized so that the sample substrate can be removed from the interior space through the at least one port.

According to various aspects of embodiments of this disclosure, the separation boundary comprises at least one of the following: perforations in, cuts into or through, or locally thinned portions of the cell culture substrate. The separation boundary can further include an attachment material between the sample substrate and the remainder of the substrate, the attachment material being configured to release from one or both of the sample substrate and the remainder of the substrate under tension. The system can further include a plurality of sample substrates. At least two of the plurality of sample substrates can be separated from each other by a portion of the remainder of the cell culture substrate that is not one of the plurality of sample substrates. At least a portion of the plurality of sample substrates can be separated from each other by the separation boundary and without any of the remainder of the cell culture substrate that is not one of the plurality of sample substrate therebetween.

The bioreactor vessel is configured for aseptic removal of the sample substrate from the interior space. The system may include a tether comprising a first end that is attached to the sample substrate, the tether being configured to pull the sample substrate out of the interior space through the port. The system may further include a capture device disposed outside of the interior space and configured to contain the sample substrate after the sample substrate is removed from the interior space via the at least one port. The capture device may have flexible walls, pleated walls, or folds that enable the capture device to have a variable interior volume. The system can further include a plug disposed along the tether between the first end and the second end of the tether, wherein the plug is configured to form an aseptic seal in the port while the sample substrate remains in the interior space. The plug is configured to dislodge from the port when the second end of the tether is pulled away from the port with a predetermined force.

### BRIEF DESCRIPTION OF THE DRAWINGS

FIG. 1A shows a perspective view of a three-dimensional model of a cell culture substrate, according to embodiments of this disclosure.

FIG. 1B is a two-dimensional plan view of the substrate of FIG. 1A.

FIG. 1C is a cross-section along line A-A of the substrate in FIG. 1B.

FIG. 2 shows a schematic view of a cell culture system, according to embodiments.

FIG. 3A shows a plan view of a cell culture substrate sample layer with a separation boundary defining a substrate sample portion, according to embodiments.

FIG. 3B shows a plan view of the cell culture substrate sample layer and sample portion of FIG. 3A after the sample portion is separated from the remainder of the substrate sample layer, according to embodiments.

FIG. 4A shows a plan view ell culture substrate sample layer with a plurality of substrate sample portions having tapered ends, according to embodiments.

FIG. 4B shows an individual substrate sample portion from FIG. 4A.

FIG. 4C shows the individual substrate sample portion of FIG. 4B and a port through which the substrate sample portion can be extracted from a fixed bed cell culture substrate.

FIG. 5A shows a sidewall of a cell culture vessel having a number of ports for accessing cell culture sample portions in the interior reservoir of the vessel, according to embodiments.

FIG. 5B shows a cross-section view of the sidewall of FIG. 5A at line A-A in FIG. 5A, according to embodiments.

FIG. 6A shows an isometric view of a bioreactor with a series of sampling reels on the side of the bioreactor, according to embodiments.

FIG. 6B is a close-up isometric view of one of the sampling reels from FIG. 6A, according to embodiments.

FIG. 7 is a cross-section view of the sampling reel from FIG. 6B, according to embodiments.

FIG. 8A is a cross-section view of the sampling reel from FIG. 7 in a state of rotation to extract a sample substrate, according to embodiments.

FIG. 8B is a cross-section view of the sampling reel from FIG. 8A after extracting the sample substrate.

FIG. 9A is a cross-section view of the sampling reel from FIGS. 7-8B after rotating the reel to align the substrate sample opening and the housing opening to enable extraction of the substrate sample, according to embodiments.

FIG. 9B is a cross-section view of the sampling reel from FIG. 9A, after a counterclockwise rotation to produce the sample substrate, according to embodiments.

FIG. 10A shows the sample substrate emerging from the substrate sample opening, according to embodiments.

FIG. 10B shows a cross-section view of a sampling reel from FIG. 10A after the sample substrate has been extracted, according to embodiments.

FIG. 11A shows the spool in a position to receive a sample substrate from the bioreactor, according to embodiments.

FIG. 11B shows the spool in a sealed position, according to embodiments.

FIG. 11C shows the spool of FIGS. 11A and 11B with the spool in position to produce the sample substrate from the reel, according to embodiments.

FIG. 12A shows an isometric view of a multi-layered cell culture substrate assembly in a sleeve with cutouts for sampling, according to embodiments.

FIG. 12B shows a plan view of the assembly in FIG. 12A.

FIG. 13A shows an isometric view of the assembly of FIG. 12A with seals along the cutouts, according to embodiments.

FIG. 13B shows a plan view of the assembly in FIG. 13A.

FIG. 14A shows an isometric view of the assembly of FIG. 13A disposed in an outer housing of a bioreactor vessel, according to embodiments.

FIG. 14B shows a plan view of the assembly in FIG. 14A.

FIG. 15 shows an isometric view of the assembly in FIG. 14A with the multi-layered cell culture substrate and sleeve lifted out of the housing of the bioreactor vessel for sampling, according to embodiments.

FIG. 16 is a plan view of a substrate sample cartridge assembly, according to embodiments.

FIG. 17 is a plan view of a substrate sample layer with tethered sample portions, according to embodiments.

FIG. 18A is a photograph of a substrate sample layer of woven PET mesh, according to embodiments.

FIG. 18B is a photograph of crystal violet stained sample portions of woven PET mesh prior to harvesting the adherent cells, according to embodiments.

FIG. 18C is a photograph of crystal violet stained sample portions of woven PET mesh substrate after harvesting the adherent cells, according to embodiments.

FIG. 19A is a cross-section view of a sample capture device attached to a side wall of a bioreactor for receiving a sample portion from within the reactor, according to embodiments.

FIG. 19B is a cross-section view of a sample capture device of FIG. 19A that has received a sample portion from within the reactor, according to embodiments.

FIG. 19C is a cross-section view showing the attachment of the sample capture device from FIGS. 19A and 19B to the bioreactor and around a tether of a sample portion, according to embodiments.

FIG. 20A is a cross-section view showing another sample capture device, according to embodiments.

FIG. 20B shows the sample capture device of FIG. 20A in the process of attaching to a tether of a sample portion in order to remove the sample portion.

FIG. 20C shows the sample capture device of FIGS. 20A and 20B after removal of the sample portion from the bioreactor and sealing of the sample capture device, according to embodiments.

FIG. 21A is a cross-section view of a sample capture device attached to a side wall of a bioreactor with a valve cam ring for sealing the port after removing the sample portion, according to embodiments.

FIG. 21B shows a cross-section view of the valve cam ring of FIG. 21A, according to embodiments.

FIG. 21C is a cross-section view of the sample capture device after removing the sample portion and closing the valve cam ring of FIGS. 21A and 21B, according to embodiments.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

Embodiments of this disclosure include cell culture substrates, as well as cell culture bioreactors incorporating such a substrate, that enabling sampling of the substrate or a portion of the substrate for monitoring cell culture.

In conventional large-scale cell culture bioreactors, different types of packed bed bioreactors have been used. Usually these packed beds contain porous matrices to retain adherent or suspension cells, and to support growth and proliferation. Packed-bed matrices provide high surface area to volume ratios, so cell density can be higher than in the other systems. However, the packed bed often functions as a depth filter, where cells are physically trapped or entangled in fibers of the matrix. Thus, because of linear flow of the cell inoculum through the packed bed, cells are subject to heterogeneous distribution inside the packed-bed, leading to variations in cell density through the depth or width of the packed bed. For example, cell density may be higher at the inlet region of a bioreactor and significantly lower nearer to the outlet of the bioreactor. This non-uniform distribution of the cells inside of the packed-bed significantly hinders scalability and predictability of such bioreactors in bioprocess manufacturing, and can even lead to reduced efficiency in terms of growth of cells or viral vector production per unit surface area or volume of the packed bed.

Another problem encountered in packed bed bioreactors disclosed in prior art is the channeling effect. Due to random nature of packed nonwoven fibers, the local fiber density at any given cross section of the packed bed is not uniform. Medium flows quickly in the regions with low fiber density (high bed permeability) and much slower in the regions of high fiber density (lower bed permeability). The resulting non-uniform media perfusion across the packed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. Cell harvesting is yet another problem encountered when bioreactors packed with non-woven fibrous scaffolds are used. Due to packed-bed functions as depth filter, cells that are released at the end of cell culture process are entrapped inside the packed bed, and cell recovery is very low. This significantly limits utilization of such bioreactors in bioprocesses where live cells are the products. Thus, the non-uniformity leads to areas with different exposure to flow and shear, effectively reducing the usable cell culture area, causing non-uniform culture, and interfering with transfection efficiency and cell release.

To address these and other problems of existing cell culture solutions, embodiments of the present disclosure provide cell growth substrates, matrices of such substrates, and/or packed-bed systems using such substrates that enable efficient and high-yield cell culturing for anchorage-dependent cells and production of cell products (e.g., proteins, antibodies, viral particles). Embodiments include a porous cell-culture matrix made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. For example, in some embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture matrix having a high surface area that, in combination with the other features described, enables a high yield cell culture solution. In some embodiments, for example, the cell culture substrate and/or bioreactors discussed herein can produce 10¹⁶ to 10¹⁸ viral genomes (VG) per batch.

In embodiments, a matrix is provided with a structurally defined surface area for adherent cells to attach and proliferate that has good mechanical strength and forms a highly uniform multiplicity of interconnected fluidic networks when assembled in a packed bed or other bioreactor. In particular embodiments, a mechanically stable, non-degradable woven mesh can be used as the substrate to support adherent cell production. The cell culture matrix disclosed herein supports attachment and proliferation of anchorage dependent cells in a high volumetric density format. Uniform cell seeding of such a matrix is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed matrix, and can avoid formation of large and/or uncontrollable 3D cellular aggregates with limited nutrient diffusion and increased metabolite concentrations. Thus, the matrix eliminates diffusional limitations during operation of the bioreactor. In addition, the matrix enables easy and efficient cell harvest from the bioreactor. The structurally defined matrix of one or more embodiments enables complete cell recovery and consistent cell harvesting from the packed bed of the bioreactor.

According to embodiments, a method of cell culturing is also provided using bioreactors with the matrix for bioprocessing production of therapeutic proteins, antibodies, viral vaccines, or viral vectors.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the packed bed. This construction enables improved cell seeding, nutrient delivery, cell growth, and cell harvesting. According to one or more particular embodiments, the matrix is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways discussed here for uniform cell seeding and growth, uniform media perfusion, and efficient cell harvest.

Embodiments of this disclosure can achieve viral vector platforms of a practical size that can produce viral genomes on the scale of greater than about 10¹⁴ viral genomes per batch, greater than about 10¹⁵ viral genomes per batch, greater than about 10¹⁶ viral genomes per batch, greater than about 10¹⁷ viral genomes per batch, or up to or greater than about g 10¹⁶ viral genomes per batch. In some embodiments, productions is about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

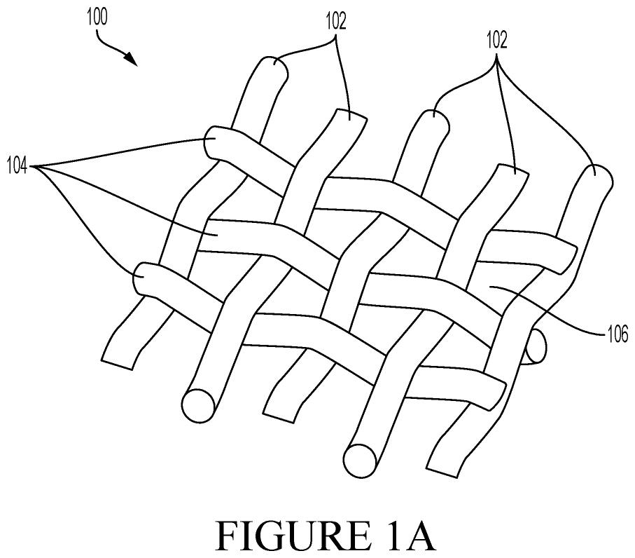

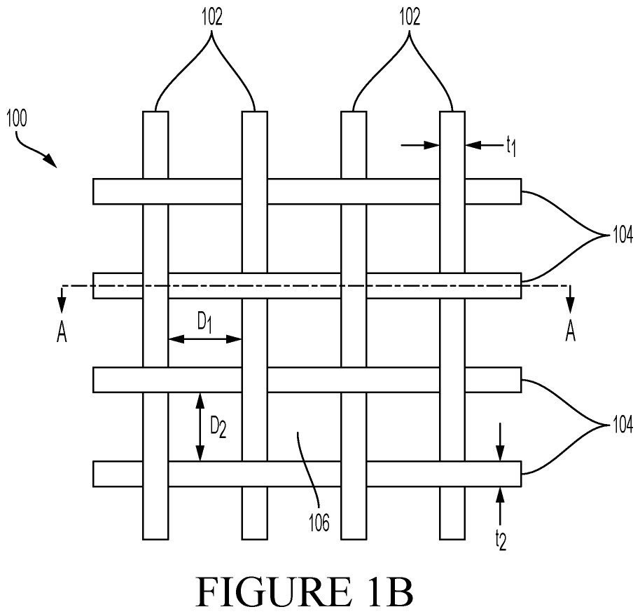

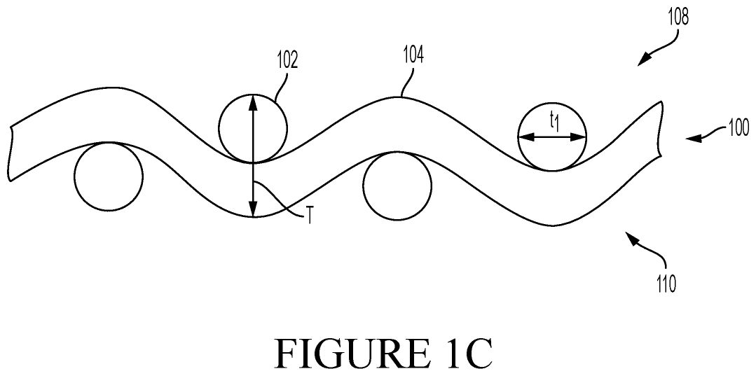

FIGS. 1A and 1B show a three-dimensional (3D) perspective view and a two-dimensional (2D) plan view, respectively, of a cell culture substrate 100, according to an example of one or more embodiments of this disclosure. The cell culture substrate 100 is a woven mesh layer made of a first plurality of fibers 102 running in a first direction and a second plurality of fibers 104 running in a second direction. The woven fibers of the substrate 100 form a plurality of openings 106, which can be defined by one or more widths or diameters (e.g., D₁, D₂). The size and shape of the openings can vary based on the type of weave (e.g., number, shape and size of filaments; angle between intersecting filaments, etc.). A woven mesh may be characterized as, on a macro-scale, a two-dimensional sheet or layer. However, a close inspection of a woven mesh reveals a three-dimensional structure due to the rising and falling of intersecting fibers of the mesh. Thus, as shown in FIG. 1C, a thickness T of the woven mesh 100 may be thicker than the thickness of a single fiber (e.g., t₁). As used herein, the thickness T is the maximum thickness between a first side 108 and a second side 110 of the woven mesh. Without wishing to be bound by theory, it is believed that the three-dimensional structure of the substrate 100 is advantageous as it provides a large surface area for culturing adherent cells, and the structural rigidity of the mesh can provide a consistent and predictable cell culture matrix structure that enables uniform fluid flow.

In FIG. 1B, the openings 106 have a diameter D₁, defined as a distance between opposite fibers 102, and a diameter D₂, defined as a distance between opposite fibers 104. D₁ and D₂ can be equal or unequal, depending on the weave geometry. Where D₁ and D₂ are unequal, the larger can be referred to as the major diameter, and the smaller as the minor diameter. In some embodiments, the diameter of an opening may refer to the widest part of the opening. Unless otherwise specified, the opening diameter, as used herein, will refer to a distance between parallel fibers on opposite sides of an opening.

A given fiber of the plurality of fibers 102 has a thickness t₁, and a given fiber of the plurality of fibers 104 has a thickness t₂. In the case of fibers of round cross-section, as shown in FIG. 1A, or other three-dimensional cross-sections, the thicknesses t₁ and t₂ are the maximum diameters or thicknesses of the fiber cross-section. According to some embodiments, the plurality of fibers 102 all have the same thickness t₁, and the plurality of fiber 104 all have the same thickness t₂. In addition, t₁ and t₂ may be equal. However, in one or more embodiments, t₁ and t₂ are not equal such as when the plurality of fibers 102 are different from the plurality of fiber 104. In addition, each of the plurality of fibers 102 and plurality of fibers 104 may contain fibers of two or more different thicknesses (e.g., t₁ₐ, t_{1b}, etc., and t₂ₐ, t_{2b}, etc.). According to embodiments, the thicknesses t₁ and t₂ are large relative to the size of the cells cultured thereon, so that the fibers provide an approximation of a flat surface from the perspective of the cell, which can enable better cell attachment and growth as compared to some other solutions in which the fiber size is small (e.g., on the scale of the cell diameter). Due to three-dimensional nature of woven mesh, as shown in FIGS. 1A-1C, the 2D surface area of the fibers available for cell attachment and proliferation exceeds the surface area for attachment on an equivalent planar 2D surface.

In one or more embodiments, a fiber may have a diameter in a range of about 50 μm to about 1000 μm; about 100 μm to about 750 μm; about 125 μm to about 600 μm; about 150 μm to about 500 μm; about 200 μm to about 400 μm; about 200 μm to about 300 μm; or about 150 μm to about 300 μm. On a microscale level, due to the scale of the fiber compared to the cells (e.g., the fiber diameters being larger than the cells), the surface of monofilament fiber is presented as an approximation of a 2D surface for adherent cells to attach and proliferate. Fibers can be woven into a mesh with openings ranging from about 100 μm×100 um to about 1000 μm×1000 μm. In some embodiments, the opening may have a diameter of about 50 μm to about 1000 μm; about 100 μm to about 750 μm; about 125 μm to about 600 μm; about 150 μm to about 500 μm; about 200 μm to about 400 μm; or about 200 μm to about 300 μm. These ranges of the filament diameters and opening diameters are examples of some embodiments, but are not intended to limit the possible feature sizes of the mesh according to all embodiments. The combination of fiber diameter and opening diameter is chosen to provide efficient and uniform fluid flow through the substrate when, for example, the cell culture matrix includes a number of adjacent mesh layers (e.g., a stack of individual layers or a rolled mesh layer).

Factors such as the fiber diameter, opening diameter, and weave type/pattern will determine the surface area available for cell attachment and growth. In addition, when the cell culture matrix includes a stack, roll, or other arrangement of overlapping substrate, the packing density of the cell culture matrix will impact the surface area of the packed bed matrix. Packing density can vary with the packing thickness of the substrate material (e.g., the space needed for a layer of the substrate). For example, if a stack of cell culture matrix has a certain height, each layer of the stack can be said to have a packing thickness determined by dividing the total height of the stack by the number of layers in the stack. The packing thickness will vary based on fiber diameter and weave, but can also vary based the alignment of adjacent layers in the stack. For instance, due to the three-dimensional nature of a woven layer, there is a certain amount of interlocking or overlapping that adjacent layers can accommodate based on their alignment with one another. In a first alignment, the adjacent layers can be tightly nestled together, but in a second alignment, the adjacent layers can have zero overlap, such as when the lower-most point of the upper layer is in direct contact with the upper-most point of the lower layer. It may be desirable for certain applications to provide a cell culture matrix with a lower density packing of layers (e.g., when higher permeability is a priority) or a higher density of packing (e.g., when maximizing substrate surface area is a priority). According to one or more embodiments, the packing thickness can be from about 50 μm to about 1000 μm; about 100 μm to about 750 μm; about 125 μm to about 600 μm; about 150 μm to about 500 μm; about 200 μm to about 400 μm; about 200 μm to about 300 μm.

The above structural factors can determine the surface area of a cell culture matrix, whether of a single layer of cell culture substrate or of a cell culture matrix having multiple layers of substrate). For example, in a particular embodiment, a single layer of woven mesh substrate having a circular shape and diameter of 6 cm can have an effective surface area of about 68 cm². The “effective surface area,” as used herein, is the total surface area of fibers in a portion of substrate material that is available for cell attachment and growth. Unless stated otherwise, references to “surface area” refer to this effective surface area. According to one or more embodiments, a single woven mesh substrate layer with a diameter of 6 cm may have an effective surface area of about 50 cm² to about 90 cm²; about 53 cm² to about 81 cm²; about 68 cm²; about 75 cm²; or about 81 cm². These ranges of effective surface area are provided for example only, and some embodiments may have different effective surface areas. The cell culture matrix can also be characterized in terms of porosity, as discussed in the Examples herein.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different patterns or weaves, including, for example knitted, warp-knitted, or woven (e.g., plain weave, twilled weave, dutch weave, five needle weave).

The surface chemistry of the mesh filaments may need to be modified to provide desired cell adhesion properties. Such modifications can be made through the chemical treatment of the polymer material of the mesh or by grafting cell adhesion molecules to the filament surface. Alternatively, meshes can be coated with thin layer of biocompatible hydrogels that demonstrate cell adherence properties, including, for example, collagen or Matrigel®. Alternatively, surfaces of filament fibers of the mesh can be rendered with cell adhesive properties through the treatment processes with various types of plasmas, process gases, and/or chemicals known in the industry. In one or more embodiments, however, the mesh is capable of providing an efficient cell growth surface without surface treatment.

Another advantage of the higher surface areas and high cell seeding or growing densities is that the cost of the embodiments disclosed herein can be the same or less than competing solution. Specifically, the cost per cellular product (e.g., per cell or per viral genome) can be equal to or less than other packed bed bioreactors.

By using a structurally defined culture matrix of sufficient rigidity, high-flow-resistance uniformity across the matrix or packed bed is achieved. According to various embodiments, the matrix can be deployed in monolayer or multilayer formats. This flexibility eliminates diffusional limitations and provides uniform delivery of nutrients and oxygen to cells attached to the matrix. In addition, the open matrix lacks any cell entrapment regions in the packed bed configuration, allowing for complete cell harvest with high viability at the end of culturing. The matrix also delivers packaging uniformity for the packed bed, and enables direct scalability from process development units to large-scale industrial bioprocessing unit. The ability to directly harvest cells from the packed bed eliminates the need of resuspending a matrix in a stirred or mechanically shaken vessel, which would add complexity and can inflict harmful shear stresses on the cells. Further, the high packing density of the cell culture matrix yields high bioprocess productivity in volumes manageable at the industrial scale.

As used herein, “structurally defined” means that the structure of the substrate follows a predetermined design and is not random. The structurally defined substrate can thus be a woven design, 3D printed, molded, or formed by some other technique known in the art that allows the structure to follow a predetermined planned structure.

As discussed herein, the cell culture substrate can be used within a bioreactor vessel, according to one or more embodiments. For example, the substrate can be used in a packed bed bioreactor configuration, or in other configurations within a three-dimensional culture chamber. However, embodiments are not limited to a three-dimensional culture space, and it is contemplated that the substrate can be used in what may be considered a two-dimensional culture surface configuration, where the one or more layers of the substrate lay flat, such as within a flat-bottomed culture dish, to provide a culture substrate for cells. Due to contamination concerns, the vessel can be a single-use vessel that can be disposed of after use.

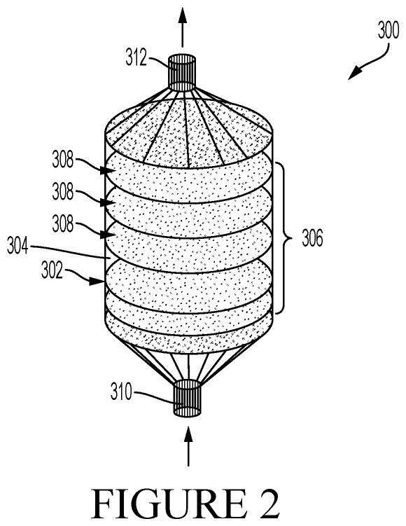

A cell culture system is provided, according to one or more embodiments, in which the cell culture matrix is used within a culture chamber of a bioreactor vessel. FIG. 2 shows an example of a cell culture system 300 that includes a bioreactor vessel 302 having a cell culture chamber 304 in the interior of the bioreactor vessel 302. Within the cell culture chamber 304 is a cell culture matrix 306 that is made from a stack of substrate layers 308. The substrate layers 308 are stacked with the first or second side of a substrate layer facing a first or second side of an adjacent substrate layer. The bioreactor vessel 300 has an inlet 310 at one end for the input of media, cells, and/or nutrients into the culture chamber 304, and an outlet 312 at the opposite end for removing media, cells, or cell products from the culture chamber 304. By allowing stacking of substrate layers in this way, the system can be easily scaled up without negative impacts on cell attachment and proliferation, due to the defined structure and efficient fluid flow through the stacked substrates. While the vessel 300 may generally be described as having an inlet 310 and an outlet 312, some embodiments may use one or both of the inlet 310 and outlet 312 for flowing media, cells, or other contents both into and out of the culture chamber 304. For example, inlet 310 may be used for flowing media or cells into the culture chamber 304 during cell seeding, perfusion, or culturing phases, but may also be used for removing one or more of media, cells, or cell products through the inlet 310 in a harvesting phase. Thus, the terms “inlet” and “outlet” are not intended to restrict the function of those openings.

In FIG. 2, the bulk flow direction of fluid flowing through the fixed bed within the bioreactor 300 is in a direction from the inlet 310 to the outlet 312, and, in this example, the first and second major sides of the substrate layers 308 are perpendicular to the bulk flow direction.

The cell culture matrix can be arranged in multiple configurations within the culture chamber depending on the desired system. For example, in one or more embodiments, the system includes one or more layers of the substrate with a width extending across the width of a defined cell culture space in the culture chamber. Multiple layers of the substrate may be stacked in this way to a predetermined height. As discussed above, the substrate layers may be arranged such that the first and second sides of one or more layers are perpendicular to a bulk flow direction of culture media through the defined culture space within the culture chamber, or the first and second sides of one or more layers may be parallel to the bulk flow direction. In one or more embodiments, the cell culture matrix includes one or more substrate layers at a first orientation with respect to the bulk flow, and one or more other layers at a second orientation that is different from the first orientation. For example, various layers may have first and second sides that are parallel or perpendicular to the bulk flow direction, or at some angle in between.

In one or more embodiments, the cell culture system includes a plurality of discrete pieces of the cell culture substrate in a packed bed configuration, where the length and or width of the pieces of substrate are small relative to the culture chamber. As used herein, the pieces of substrate are considered to have a length and/or width that is small relative to the culture chamber when the length and/or width of the piece of substrate is about 50% or less of the length and/or width of the culture space. Thus, the cell culture system may include a plurality of pieces of substrate packed into the culture space in a desired arrangement. The arrangement of substrate pieces may be random or semi-random, or may have a predetermined order or alignment, such as the pieces being oriented in a substantially similar orientation (e.g., horizontal, vertical, or at an angle between 0° and 90° relative to the bulk flow direction).

The “defined culture space,” as used herein, refers to a space within the culture chamber occupied by the cell culture matrix and in which cell seeding and/or culturing is to occur. The defined culture space can fill approximately the entirety of the culture chamber, or may occupy a portion of the space within the culture chamber. As used herein, the “bulk flow direction” is defined as a direction of bulk mass flow of fluid or culture media through or over the cell culture matrix during the culturing of cells, and/or during the inflow or outflow of culture media to the culture chamber.

In one or more embodiments, the cell culture matrix is secured within the culture chamber by a fixing mechanism. The fixing mechanism may secure a portion of the cell culture matrix to a wall of the culture chamber that surrounds the matrix, or to a chamber wall at one end of the culture chamber. In some embodiments, the fixing mechanism adheres a portion of the cell culture matrix to a member running through the culture chamber, such as member running parallel to the longitudinal axis of the culture chamber, or to a member running perpendicular to the longitudinal axis. However, in one or more other embodiments, the cell culture matrix may be contained within the culture chamber without being fixedly attached to the wall of the chamber or bioreactor vessel. For example, the matrix may be contained by the boundaries of the culture chamber or other structural members within the chamber such that the matrix is held within a predetermined area of the bioreactor vessel without the matrix being fixedly secured to those boundaries or structural members.

The bioreactor vessel optionally includes one or more outlets capable of being attached to inlet and/or outlet means. Through the one or more outlets, liquid, media, or cells can be supplied to or removed from the chamber. A single port in the vessel may act as both the inlet and outlet, or multiple ports may be provided for dedicated inlets and outlets.

The packed bed cell culture matrix of one or more embodiments can consist of the woven cell culture mesh substrate without any other form of cell culture substrate disposed in or interspersed with the cell culture matrix. That is, the woven cell culture mesh substrate of embodiments of this disclosure are effective cell culture substrates without requiring the type of irregular, non-woven substrates used in existing solution. This enables cell culture systems of simplified design and construction, while providing a high-density cell culture substrate with the other advantages discussed herein related to flow uniformity, harvestability, etc.

As discussed herein, the cell culture substrates and bioreactor systems provided offer numerous advantages. For example, the embodiments of this disclosure can support the production of any of a number of viral vectors, such as AAV (all serotypes) and lentivirus, and can be applied toward in vivo and ex vivo gene therapy applications. The uniform cell seeding and distribution maximizes viral vector yield per vessel, and the designs enable harvesting of viable cells, which can be useful for seed trains consisting of multiple expansion periods using the same platform. In addition, the embodiments herein are scalable from process development scale to production scale, which ultimately saves development time and cost. The methods and systems disclosed herein also allow for automation and control of the cell culture process to maximize vector yield and improve reproducibility. Finally, the number of vessels needed to reach production-level scales of viral vectors (e.g., 10¹⁶ to 10¹⁸ AAV VG per batch) can be greatly reduced compared to other cell culture solutions.

It is desirable to be able to monitor bioreactors used to culture cells and/or make virus products and/or used to create a seed train to facilitate cell expansion for biochemical production. When using adherent cells in bioreactors, samples of the growth media will not contain cells, so the cell cultures or cell number cannot be measured by the out-going media. To be able to estimate the number of cells and their distribution in an adherent cell culture fixed bed, one method is to remove a portion of the substrate in the midst of the cell expansion process, according to embodiments of this disclosure. By sampling during the cell culture process, information about the run can be used to assess the quality and performance of the culture process. Cell count can be estimated from the sample and growth can be monitored by sampling at different times. This information can be used to develop and optimize performance of specific biological processes such as seed train and viral vector production. In production, runs that are contaminated or out of specification can be terminated to reduce the cost of running the process to its end without a satisfactory result. Growth media and lost production time represent significant cost for typical biological processes.

Thus, this disclosure describes substrates and methods to cut and perforate layers of cell culture substrate, including polymer mesh substrates, to create a detachable sample portion. The disclosure also describes methods and apparatus to aseptically remove the sample from a bioreactor. By sampling during the cell culture process, information about the run can be used to assess the quality and performance of the culture process. Cell count can be estimated from the sample and growth can be monitored by sampling at different times or at different places within the bioreactor. This information can be used to develop and optimize parameters for specific biological processes such as seed train and viral vector production. In production, processes that are contaminated or out of specification, can be terminated to reduce the cost of running the process to its end without a satisfactory result. Growth media and lost production time represent significant cost for typical biological processes.

In embodiments herein, substrate sample portions are separable from a remainder of the cell culture substrate with a low force to allow sampling to be accomplished ideally by hand and without disturbing the main mesh body during the sampling process. For example, a relatively low (e.g., applied by hand) force can cause the sample portion to separate from the remainder of the substrate via the tension between the sample portion to which the force is applied and the remainder of the substrate. To keep the removal force low, the separation boundary can be applied between the sample portion and the remainder of the substrate. This separation boundary can be formed, for example, by scoring, perforation, laser cutting, or other cutting means such as die cutting, and can be used to create a layer of substrate that includes separable pieces of the substrate that can be removed from the fixed bed.

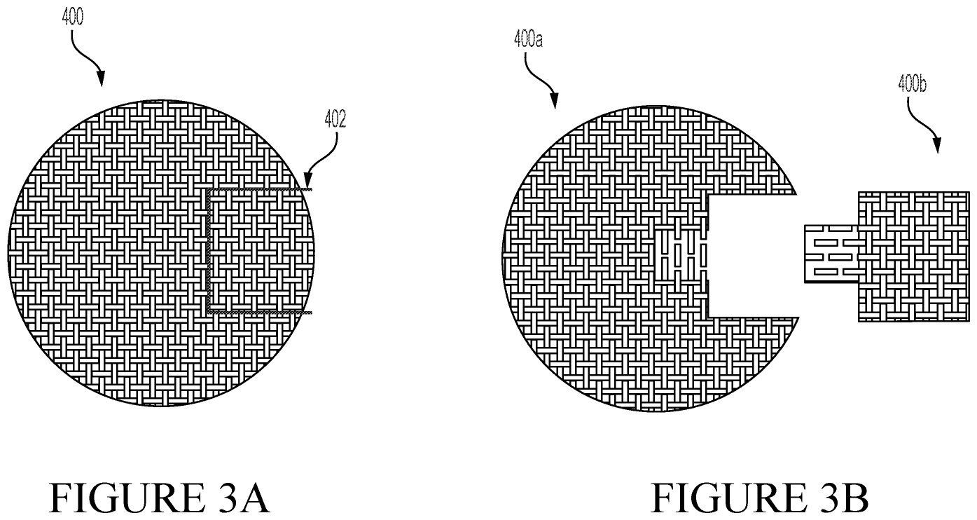

Some embodiments use woven polymer mesh substrates that have woven fibers defining an ordered array of pores or openings. Because each of the fibers in the mesh can be very strong, it is sometimes desirable to have no fibers that run between the detachable sample and the main body of the mesh to facilitate the sample being removed with a low force. It can also be desirable to have the mesh layer be robust when handled during the manufacturing and assembly process used to create a mesh stack bioreactor bed. To accomplish this, some fibers can be cut in such a way to leave a woven portion of the mesh that connects the sample to the main mesh body as shown in FIG. 3A. Due to the relative stiffness of the fibers, which may be formed from a variety of polymers disclosed herein (including PET), the interwoven fibers may remain attached even though individual fibers are severed to create the separation boundary of the sample portion. The lines in FIG. 3A show the separation boundary 402. FIG. 3B shows the sample portion after it has detached from the remainder of the cell culture substrate.

Mesh layers with sample pieces cut can be removed from bioreactors by opening the bioreactor housing and pulling them off the bed with a sterile tool or they can be removed from the reactor by using an aseptic sampling port.

A multiplicity of sampling pieces can come from a single layer. In embodiments using woven substrates, the direction of the warp and the weave as it interacts with the cutting pattern for most cutting patterns is considered to maintain the structural integrity and enable easy removal. Some cutting patterns created are less sensitive to the orientation of the mesh fibers and these patterns are advantages to use in manufacturing because the mesh orientation does not need to be precisely controlled.

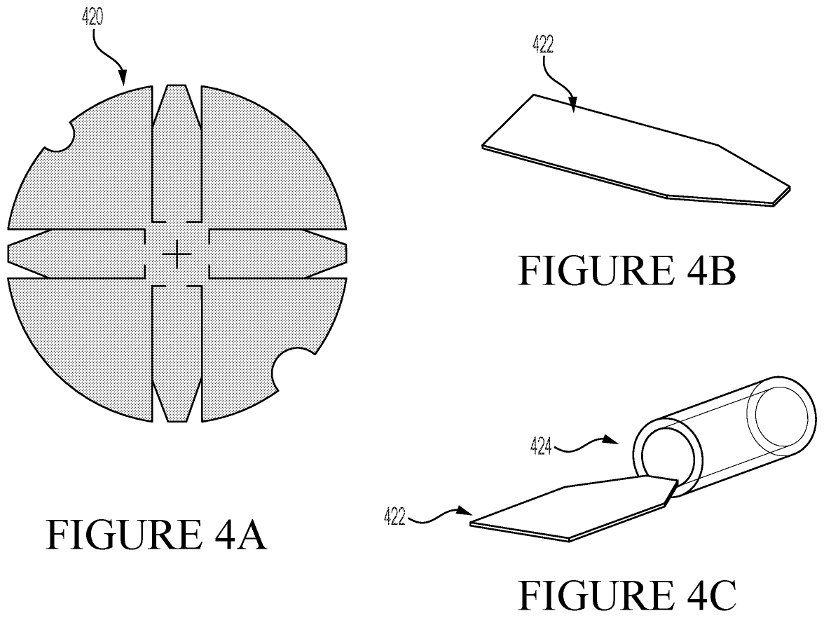

FIGS. 4A-4C show an example of embodiments in which the sampling layer contains multiple sampling portions. The shape of the sampling portions includes a rectangular end on the interior side of the sampling portion within the periphery of the sampling layer, and a tapered end on the exterior end at the periphery of the sampling layer. The tapered end allows for easy removal of the sampling portion through a port 424 in the sidewall of the bioreactor. The substrate material is such that the size of the port in the sidewall can be at or just larger than the side of the narrow end, and the wider portion of the sampling portion can slightly fold or curve as it is pulled through the opening in the sidewall. FIG. 4B shows a close up view of an individual sampling portion 422 after being detached, and FIG. 4C shows an example of the relative size between the sidewall port 424 in the bioreactor and the sample portion 422, although the relative sizes can vary in various embodiments.

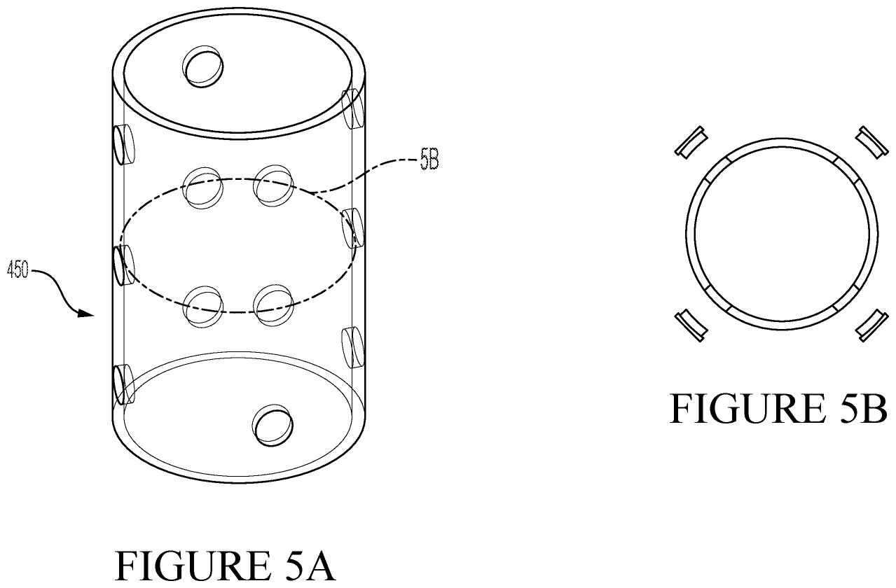

An aseptic port assembly can be modular so sampling locations can be added to the reactor at any elevation and orientation. FIG. 5A shows three layers of four sample ports assembled to a bioreactor vessel. FIG. 5b is a plan view through line A-A in FIG. 5A, which an exploded view of the port fittings in the sidewall of the bioreactor. These port fittings can be used to attach aseptic capture mechanisms on the exterior of the vessel for capturing the sample portions and maintaining them in an aseptic environment. Embodiments of this disclosure include apparatuses and methods for aseptically taking samples from the cell culture bioreactor during the course of cell culture. Embodiments include reels or rotational drums connected to the ports on the sidewall of the bioreactor. By rotating these reels, sample substrates can be aseptically extracted from the bioreactor and then extracted from the reels for analysis of cell count and culture progress and/or health.

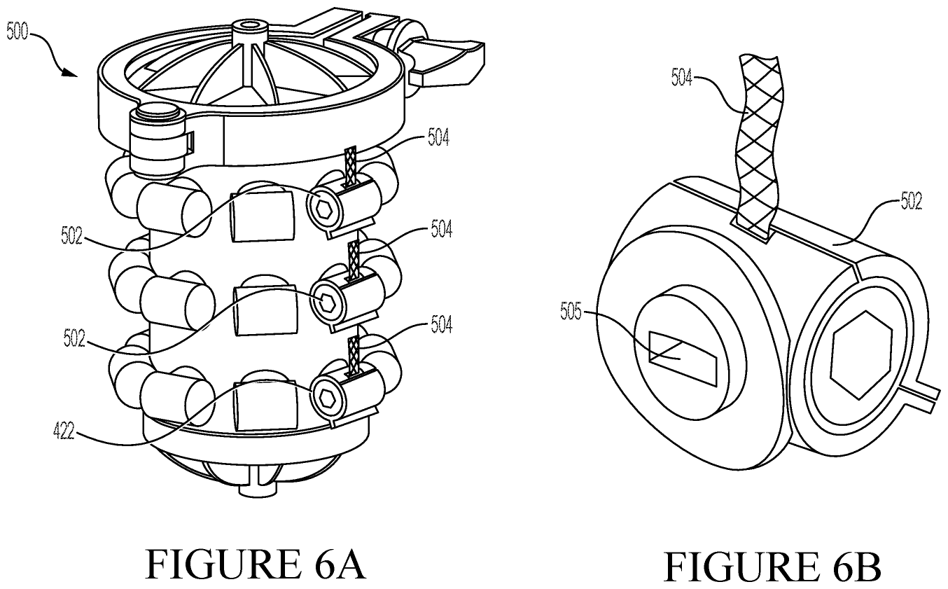

FIG. 6A shows an isometric view of a cell culture bioreactor 500 with a series of sampling reels 502 on the side of the bioreactor 500, according to embodiments. In the state shown in FIG. 6A, a number of substrate samples 504 are extending from the sampling reels 502, indicating that the substrate samples 504 have been extracted from the interior of the bioreactor vessel by the reels, and are ready for collection and analysis. FIG. 6B is a close-up view of one of the sampling reels 502 from FIG. 6A, according to embodiments. For clarity, the bioreactor vessel wall is not shown in FIG. 6B, but the passthrough opening 505 to the port on the bioreactor can be seen. It is through the passthrough opening 505 that substrate samples pass from the bioreactor vessel and into the sampling reel 502.

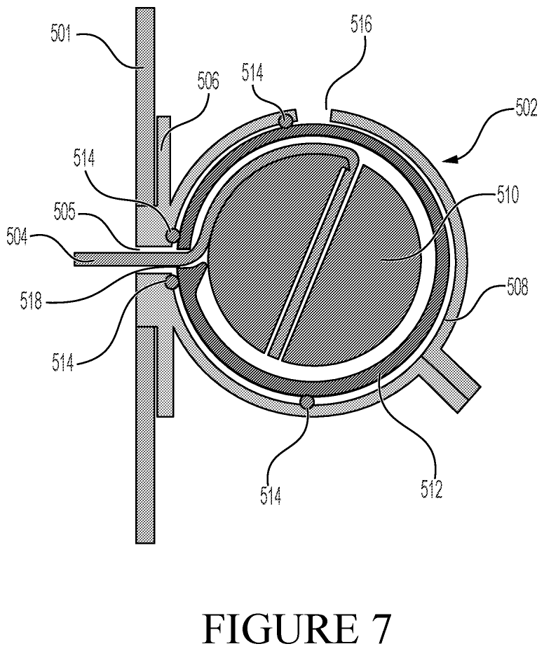

Additional details of the sampling reel 502 are shown in the cross-section view in FIG. 7. The sampling reel 502 is affixed to the bioreactor vessel wall 501 via a saddle housing 506 such that the passthrough opening 505 of the sampling reel 502 is aligned with a port on the bioreactor vessel wall 501. A remainder of the outers shell of the sampling reel 502 is made of the housing cap 508. A sample opening 516 is left between the housing cap 508 and saddle housing 506, and substrate samples will arise from this sample opening 516 (as shown in FIG. 6A). Within the sampling reel 502 is a spool 510 configured to grip a portion of sample substrate 504 such that when the spool 510 rotates, the sample substrate 504 is wound around the spool 510. The sampling reel 502 also includes a sample drum 512 disposed between the spool 510 and the outer housing (saddle housing 506 and housing cap 508) of the sampling reel 502. A series of stationary o-rings 514 are affixed to the saddle housing 506 and create seals in the space between the saddle housing 506 and the sample drum 512, to enable the aseptic sampling of the sample substrate 504. The sample drum 512 also includes a drum opening 518 to allow the sample substrate to pass within the sample drum 512 to the spool 510. As discussed below, the drum opening 518 will also enable removal of the sample substrate from the sampling reel 502.

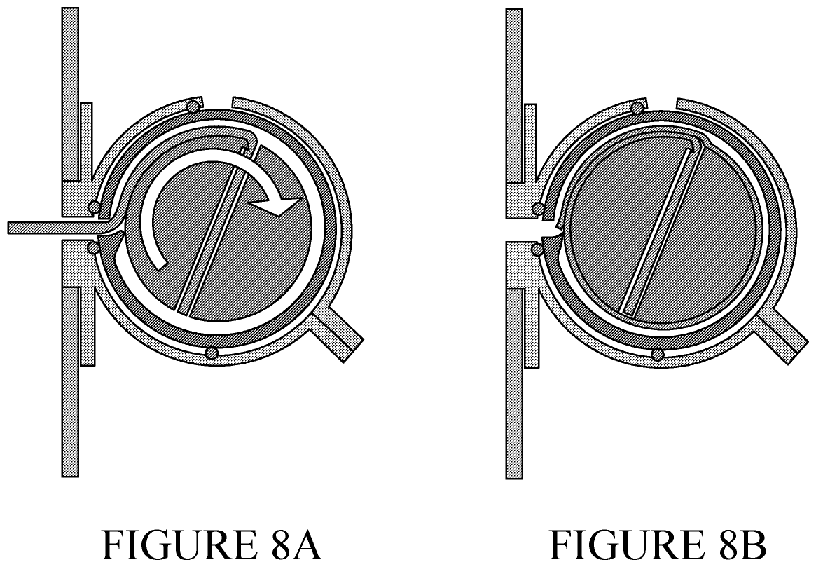

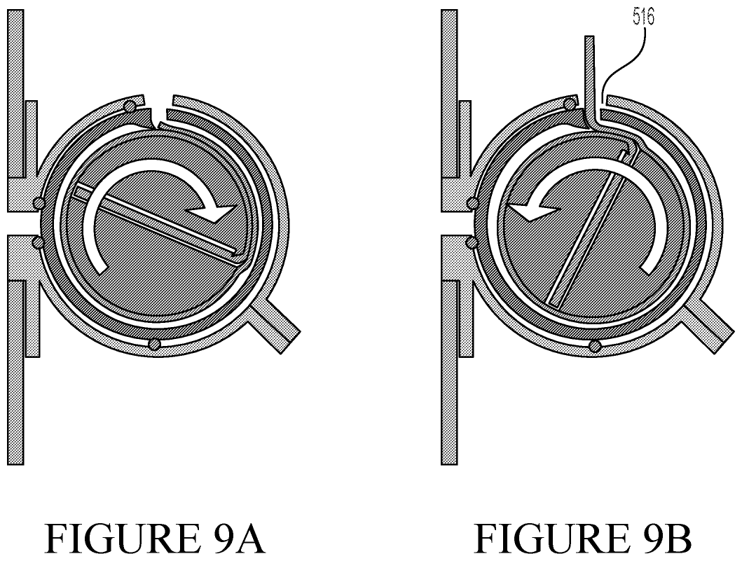

FIG. 8A is a cross-section view of the sampling reel 502 from FIG. 7 as it is rotated clockwise (as seen on the page) to extract a sample substrate 504 through the passthrough opening 505, according to embodiments. As shown in FIG. 8B, after the sampling reel 502 is rotated sufficiently to pass the entirety of the sample substrate 504 through the passthrough opening 505, the sample substrate 504 is then fully extracted from the interior of the bioreactor vessel and retained in the sampling reel 502. As shown in FIG. 9A, further turning in the same direction will align the substrate sample opening 516 with passthrough opening 505 to enable extraction of the substrate sample 504, according to embodiments. To extract the sample substrate 504, the spool 510 is then rotated in the opposite direction (counterclockwise on the page of FIG. 9B). The sample drum 512 stays in place during the reverse rotation and the substrate sample 504 is pushed out of the sampling reel 502 through the passthrough opening 505 and sample opening 516. The lifting of the substrate sample 504 through these openings can be aided by a curved or wedge surface on the edge of the passthrough opening 505, as shown in FIG. 9B.

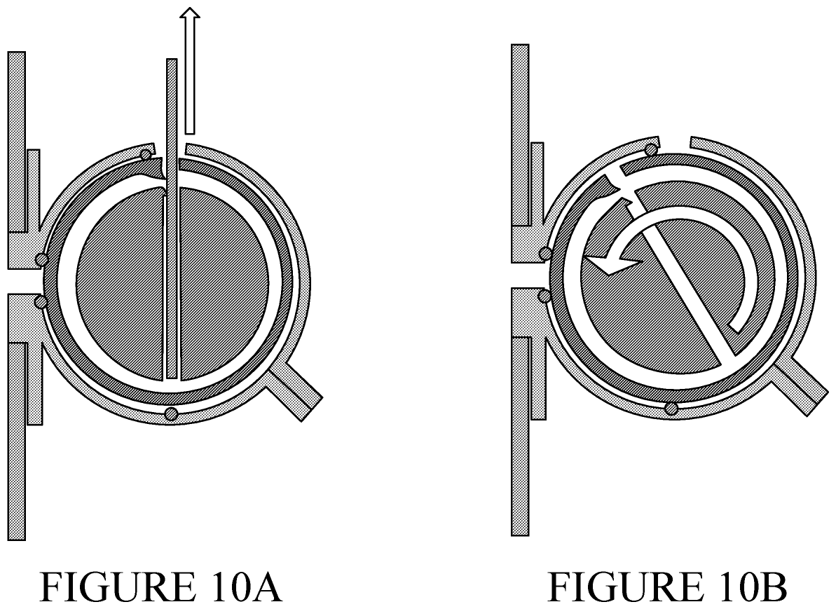

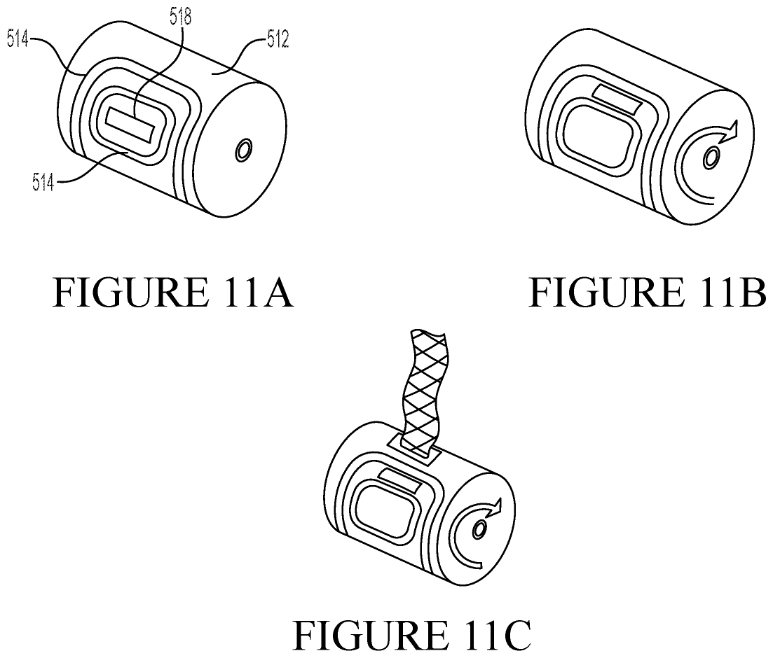

The sample drum 512 rotated further until the entire substrate sample 504 can be lifted from the sampling reel 502, as shown in FIGS. 10A and 10B. FIG. 10B also shows that the sample drum 512 can be rotated further in the counterclockwise direction (on the page of FIG. 10B) to seal the interior of the sampling reel 502 by the drum opening 518 passing the stationary o-ring 514 near the sample opening 516. Thus, the entire mechanism can deliver the substrate sample aseptically without exposing the interior of the bioreactor vessel. For clarity, FIGS. 11A-11C show isometric views of the sample drum 512 and stationary o-rings 514, without the housing cap 508 and saddle housing 506 so that it can be appreciated how the drum opening 518 moves past the series of o-rings on its way to alignment with the sample opening 516 in FIG. 11C.

According to embodiments discussed herein, aseptically sampling can be performed at any location in a cell culture fixed bed substrate. After being removed from the bioreactor, the sample portion can then, under proper conditions, be analyzed. This process limits exposure of the main cell culture to potential contamination that would be more likely if sample required opening up the entire bioreactor and removing some portion of the substrate, which might also physically disturb the cells on the portion of the cell culture that is reinserted into the bioreactor.

According to embodiments of this disclosure, a multi-layered cell culture substrate assembly is provided that allows for easy sampling of layers at defined positions along the height of the assembly. In embodiments, the cell culture substrate stack is disposed in a sleeve with predefined sample access slots. The cell culture substrate stack can be lifted from the vessel housing while still in the sleeve and layers of substrate can be removed from the assembly through the predefined sample access slots. Aspects of embodiments also include seals on the sleeve adjacent to the sample access slots to prevent bypass flow in the vessel around the substrate bed. The closure of the sleeve can be adjustable to account for variable diameter substrate layers.

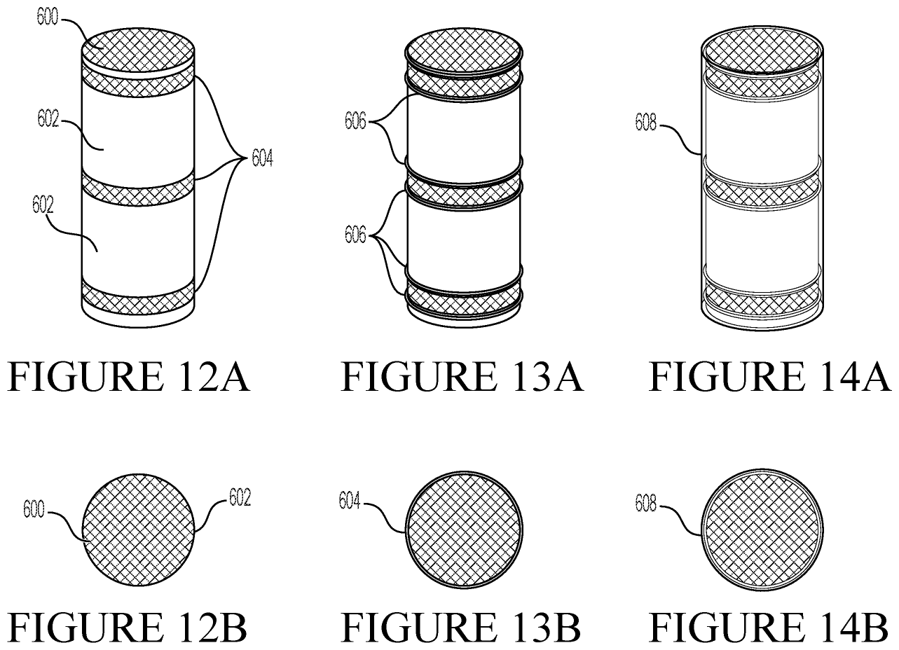

FIG. 12A shows an isometric view of a multi-layered cell culture substrate assembly in a sleeve with cutouts for sampling, according to embodiments. The assembly includes a multi-layered stack of substrate layers 600, and a sleeve 602 wrapped around the substrate layers, with predefined sample access slots 604 along the height of the assembly. By spacing the sample accesses slots 604 along the height of the assembly, samples can be taken from various positions within the fixed bed (e.g., the bottom, middle, and top) to account for possible variations due to the different positions in the packed bed. FIG. 12B shows a plan view of the assembly in FIG. 12A. FIG. 13A shows an isometric view of the assembly of FIG. 12A with the addition of seals 604 along the predefined sample access slots, according to embodiments. FIG. 13B shows a plan view of the assembly in FIG. 13A. FIG. 14A shows an isometric view of the assembly of FIG. 13A disposed in an outer housing 608 of a bioreactor vessel, according to embodiments. The seals 604 can seal the space between the sleeve 602 and the outer housing 608 to prevent bypass fluid flow from flowing out of the sample access slots and around the cell culture substrate. The seals 604 may for an interference fit with the outer housing 608, or there may be a groove or gasket to form an interlocking fit with the seals 606. FIG. 14B shows a plan view of the assembly in FIG. 14A.

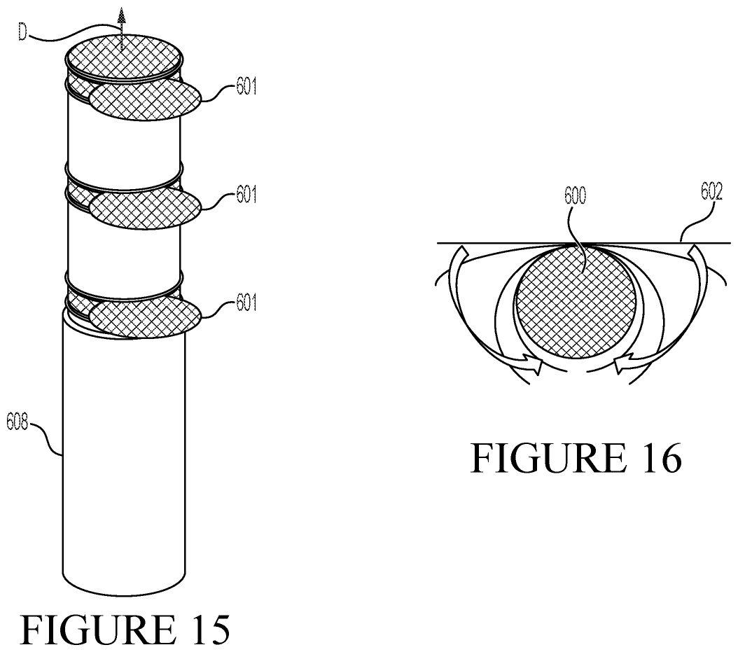

As shown in FIG. 15, when a sample needs to be collected from the assembly in FIG. 14A, the multi-layered cell culture substrate layers 600 and sleeve 602 can be lifted out of the outer housing 608 so that the sample access slots 604 can be accessed and sample substrate layers 601 can be removed via the sample access slots 604 for sampling, according to embodiments. After any sample substrate layers 601 are collected, the assembly can be reinserted into the outer housing and cell culture can continue.

FIG. 16 is a plan view of a substrate sample 600 and the sleeve 602 that can wrap around the substrate, according to embodiments. As an aspect of embodiments, the sleeve 602 may form the sample access slots by not wrapping entirely around the substrate at the predefined locations. Thus, the sample access slots 604 are windows formed by the sleeve 602 and can be sized to provide sufficient holding of the substrate layers within the window, while still allowing the substrates to be removed during sampling. For example, the opening of the sample access slots 604 or windows can be slightly narrower than a maximum width of the substrate layers.

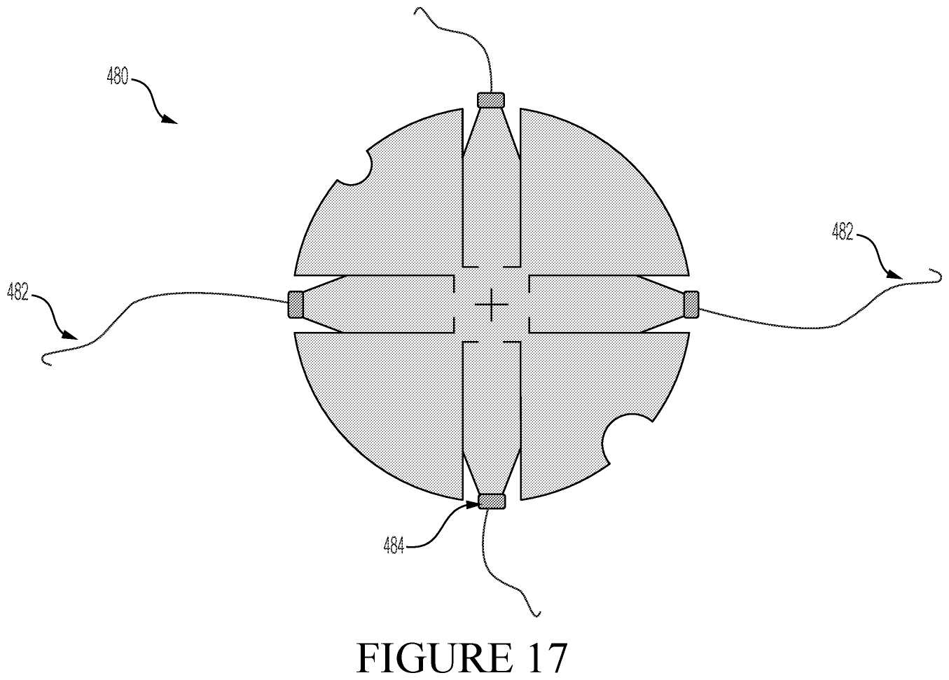

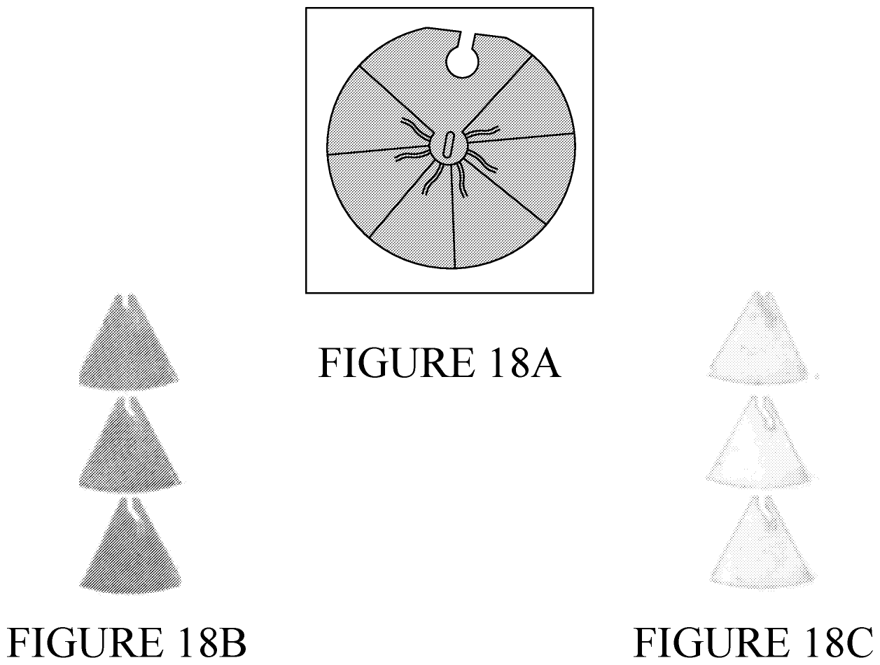

FIG. 17 shows an example where sample layers of substrate 480 have tethers 482 molded (484) to the sample portions, such that the tethers 482 can be pulled to remove the sample portions. Embodiments include a method of assembling a bioreactor in which layers of substrate are added to the bioreactor housing until the sampling port elevation is reached. At this point a sampling layer is inserted into the bioreactor vessel and the tethers are pulled through the ports. Aseptic containers on the exterior of the ports can be used to allow aseptic sampling.

FIG. 18A shows a sample layer having six pie-shaped sampling portions. The number and shape of the sampling portions can vary. In this case, the separation boundary is laser cut through the fibers of the woven mesh substrate. The sample layer also includes an alignment feature on the left side of the layer, which can be useful for keeping the sample layer in a predetermined position so that the sampling portions are in a predetermined position for easy sampling. The alignment feature can be designed to mate with a corresponding feature on the interior of the vessel sidewall. FIG. 18B shows three pie-shaped sample portions that have been stained to show the presence of adherent cells on the substrate. FIG. 18C shows three pie-shaped sample portions that have been sampled after a harvesting procedure to harvest the cells from the substrate. Comparing FIGS. 18B and 18C, shows the effectiveness of the harvesting procedure in this example.

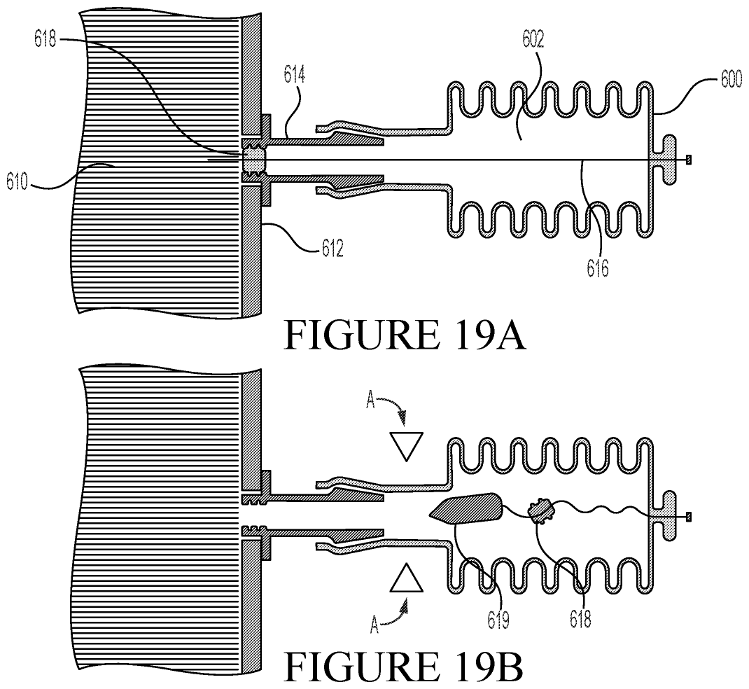

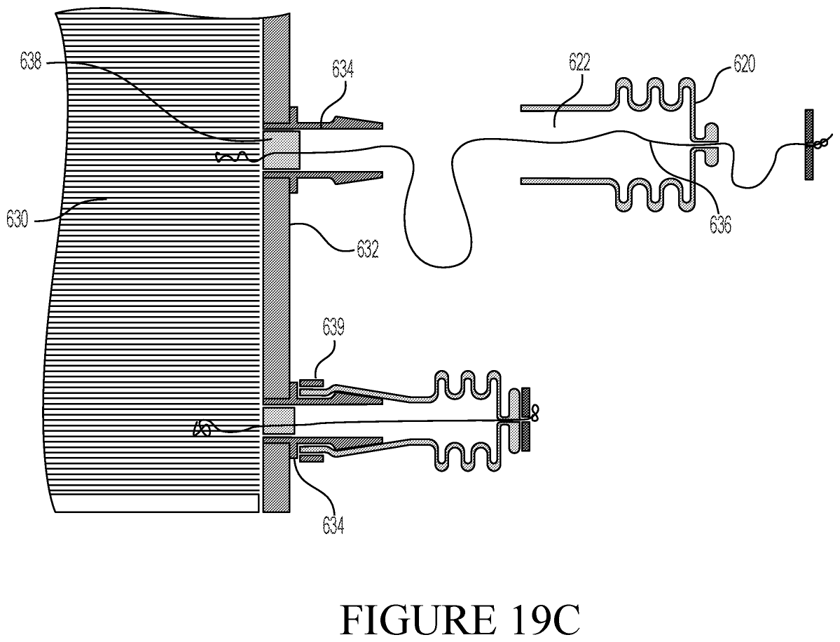

FIG. 19A shows a cross-section of a sample capture device 600, according to embodiments. The sample capture device 600 has an interior space 602 for aseptically capturing and holding a sample portion of a cell culture substrate 610 within that is held within a bioreactor 612. The capture device 600 is attached to a port 614 through which a tether 616 passes into the interior of the bioreactor 612 where it attaches to a sample portion. A seal 618 or over-mold can be disposed within the port at the entrance to the bioreactor 612 to block fluid from flowing out through the port 614 while the bioreactor is in use. An opposite side of the tether 602 can be disposed within the capture device 600, or attach to an end of the capture device 600 opposite to the port 614. When it is desired to take a sample, the end of the capture device, where the tether 602 is attached, can be puled until the tether 602 pulls the sample portion 619 (FIG. 19B) out of the bioreactor 612 and the sample portion 619 is disposed in the capture device 600. According to embodiments, the substrate layer being sample can be scored in accordance with the embodiments discussed above, so that the sample portion 619 can be easily removed. The capture device 600 can be made of a flexible and/or deformable material, such as polymer film or bag. In embodiments, the capture device 600 may resemble a “bellows” device with folds or pleats. When the bellow is extended the plug is removed and the mesh is pulled by the tether into the bellows. Using these types of capture devices for aseptic sampling, a portion of the substrate can be removed from the edge of the fixed bed substrate at any elevation in the bed without risking contamination of the bulk cell culture. A design that allows a sample portion of the fixed bed to be easily removed from the housing gives the user access to the bed without destroying the bed or bioreactor vessel. This allows any portion of the bed to be assessed. The housing can also be cleaned and reused.

FIG. 19B shows a cross-section of the sample capture device 600 during capture of the sample portion. After removal of the sample portion 619 from the bioreactor, and in order to maintain the aseptic nature of the sample taking, a portion of the sample capture device 600 near the port 614 can be sealed by a close mechanism built into the sample capture device 600 or by applying one or both of force and heat to seal the sides of the capture device together, as indicated by arrows A (e.g., by using an RF or heat tube sealer). Other means of sealing the port are also contemplated, according to embodiments, including a cap, collapsible port, sliding or hinged cover, or other means suitable to close the port and maintain the integrity of the cell culture within the bioreactor.

FIG. 19C shows a cross-section view of a bioreactor 632, according to embodiments. In this view, two aseptic sampling ports 634 are shown, although it should be appreciated that the bioreactor 632 can contain additional ports that are not shown in this view, both along the height of the bioreactor and around the circumference of the bioreactor, so that users can sample from a variety of locations in the cell culture matrix. In the process of assembling the bioreactor 632 with the cell culture substrate 630, layers of the substrate can be stacked within the bioreactor 632 until the height of a port 634 is reached. At that point, a substrate layer that is specifically designed to be a sampling substrate (e.g., with scored sample portions, as discussed above) can be inserted into the stack and a tether 636 can be fed through the port 634, into the space 622 within capture device 620, and secured to an opposite end of the capture device 620. The tether 636 can be secured by any suitable means, including by an adhesive, by tying the tether to a secure location on the capture device, by passing the tether through a hole in the opposite end of the capture device and securing the tether to a cap or stop that maintains the aseptic environment within the space 622 of the capture device 620, or by some combination of the aforementioned. For example, sealant can be applied to the hole through which the tether 636 passes in the opposite end of the capture device 620, and where the tether 636 is tied to a washer or other stop. The open end of the capture device 620 can then be attached to the port 634 by any suitable means 639, such as a clamp, band, nut, adhesive, thermal seal, or other means.

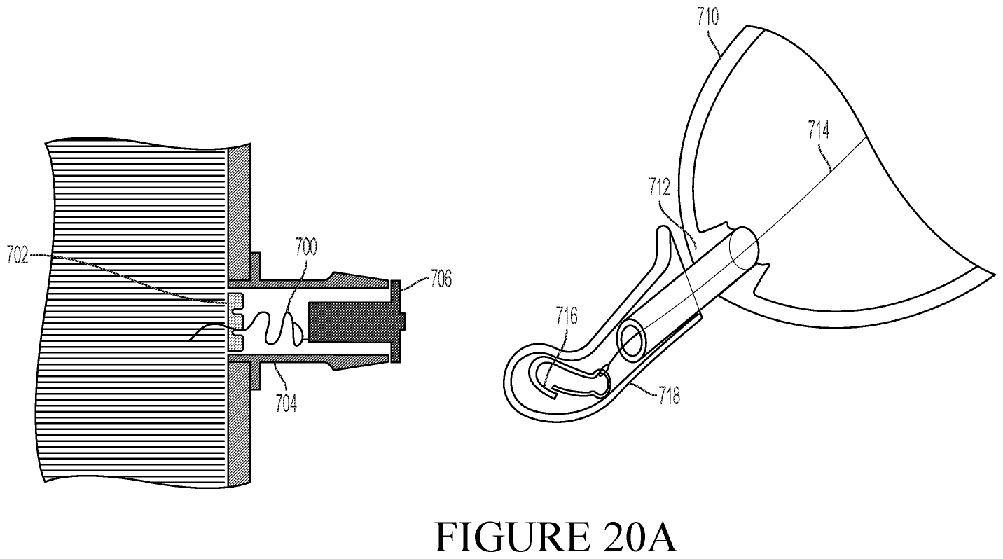

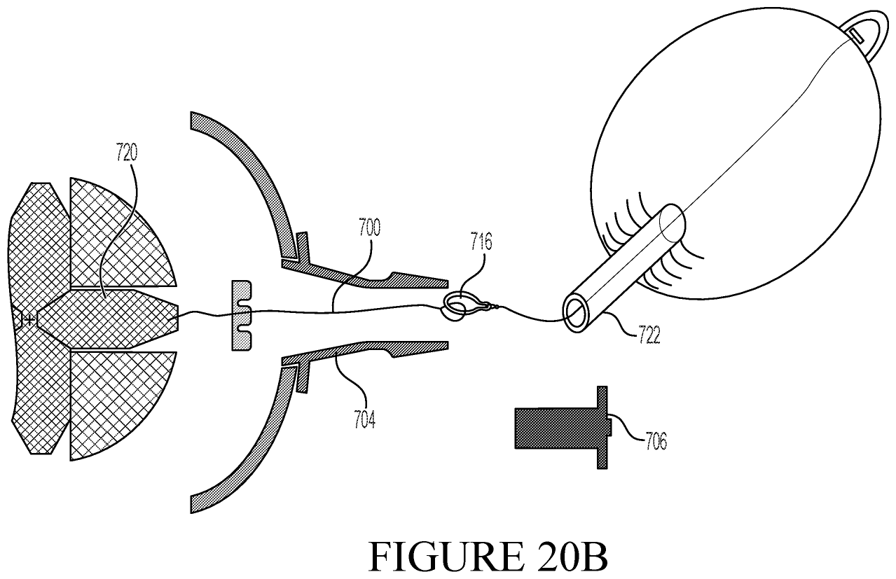

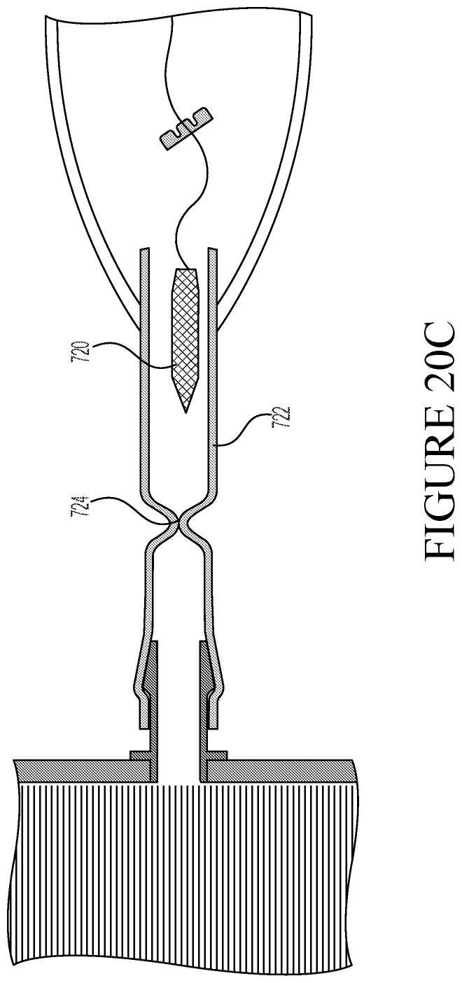

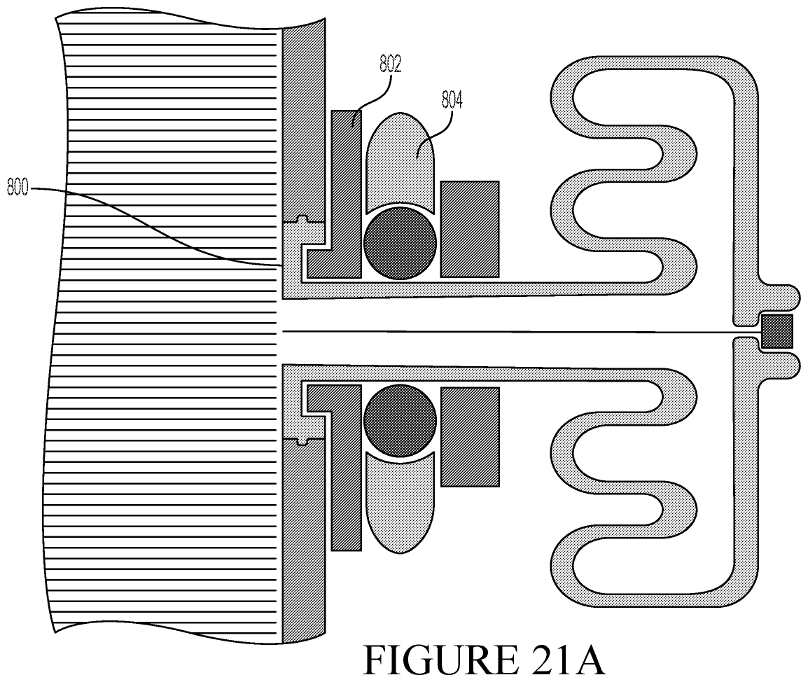

FIGS. 20A-20C show additional aspects of a capture device, according to embodiments. In FIG. 20A, a tether 700 is attached to a sampling mesh layer during assembly. The tether 700 is over-molded with a plug 702, and the port 704 is closed with a cap or plug 706 until a sample is to be taken. A sample capture device 710 is maintained separate from the bioreactor. Similar to the example in FIGS. 19A-19C, the capture device 710 has opening 712 through which a second tether 714 passes. Near the opening 712, the second tether 714 is equipment with a clasp 716 or other means for attaching to the tether 700 when the cap 706 is removed and a sample is to be taken. A protective cover 718 can cover the clasp 716 and opening 712 to maintain an aseptic environment within the capture device 710 until the sample is taken. As shown in FIGS. 20B and 20C, when taking the sample portion 720, the cap 706 is removed, the clasp 716 attached to the tether 700, and a tube 722 of the capture device 700 is attached to the port 704. After the sample portion 720 is removed and is within the tube 722 or capture device 700, the tube 722 can be sealed 724, as discussed above.

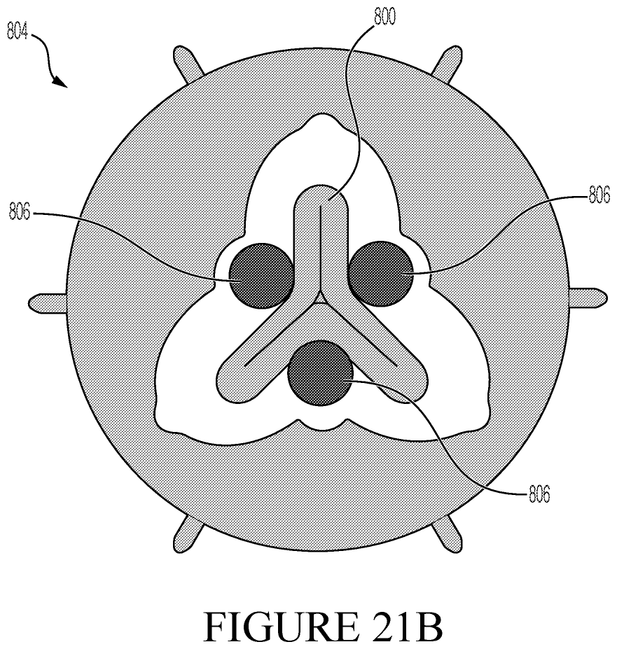

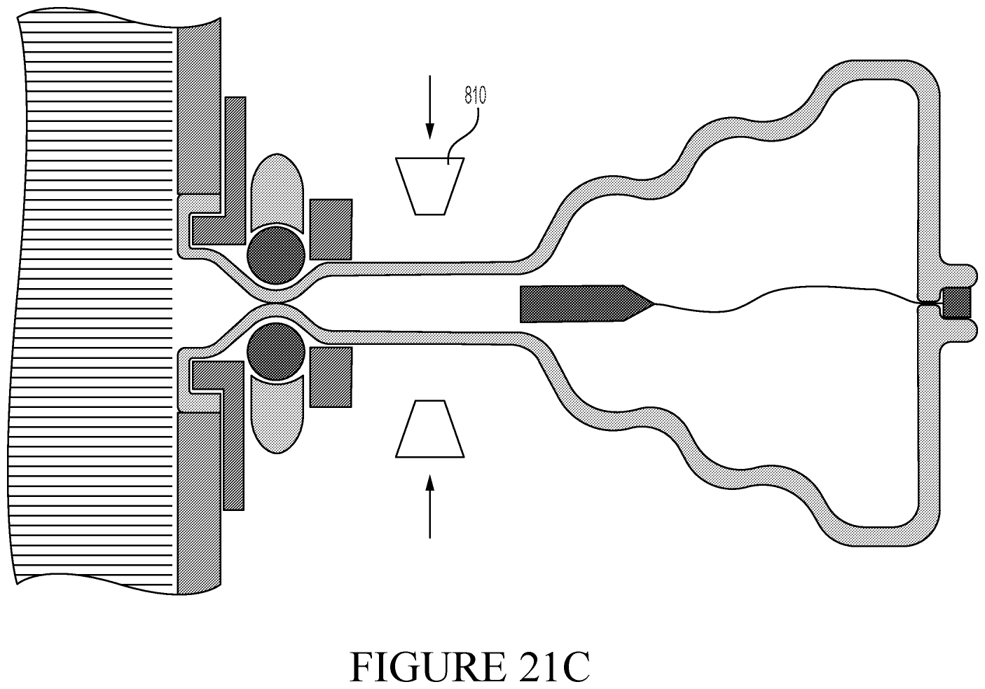

FIGS. 21A-21C show another example of a sealing means, according to embodiments of this disclosure, for closing the port on a bioreactor when not taking a sample. The configuration of the capture device and bioreactor in FIG. 21A is similar to that shown in FIG. 19A and identical features will not be described again for the sake of brevity. FIG. 21A differs from FIG. 19A in that the capture device 800 extends to the interior of the bioreactor and is attached to the port 802 at the inside wall of the bioreactor. In addition, a valve cam ring 804 is disposed around a part of the capture device 800 within the port 802. FIG. 21B shows a cross-section view of the valve cam ring 804. The valve cam ring 804 works by optionally pinching or opening the part of the capture device 800 passing through the valve cam ring 804. Inside the valve cam ring 804 are ball bearings 806 that crimp the tube of the capture device 800 shut when the valve cam ring is rotated in one direction due to the cam surfaces and detents within the valve cam ring 804, and that release the tube of the capture device 800 when rotated in the opposite direction. Therefore, the valve cam ring 804 is opened to remove the sample portion, then closed after the sample portion is disposed within the capture device 800, as shown in FIG. 21C. Optionally, the tube of the capture device 800 can then be sealed by a tube sealer 810 to provide an extra seal to protect the cell culture.

According to embodiments discussed herein, the capture device provides a way to aseptically sample a sample portion of the cell culture substrate. After being removed from the bioreactor, the sample portion can then, under proper conditions, be removed from the capture device for analysis. This process limits exposure of the main cell culture to potential contamination that would be more likely if sample required opening up the entire bioreactor and removing some portion of the substrate, which might also physically disturb the cells on the portion of the cell culture that is reinserted into the bioreactor.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.
“Include,” “includes,” or like terms means encompassing but not limited to, that is, inclusive and not exclusive.
“Users” refers to those who use the systems, methods, articles, or kits disclosed herein, and include those who are culturing cells for harvesting of cells or cell products, or those who are using cells or cell products cultured and/or harvested according to embodiments herein.
“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.
“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

### Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a fixed bed bioreactor assembly for culturing cells, the assembly comprising: a bioreactor vessel comprising an interior space for culturing cells and a sidewall at least partly defining the interior space; a plurality of cell culture substrate layers disposed in the interior space, each layer having a structurally defined surface for culturing cells thereon, the structurally defined surface defining an ordered and regular array of openings through a thickness of the layer; and a sleeve at least partially surrounding the plurality of cell culture substrate layers and comprising at least one sample access window, the sample access window comprising an opening in the sleeve configured to allow one or more layers of the cell culture substrate to be removed from the sleeve through the opening.

Aspect 2 pertains to the fixed bed bioreactor assembly of Aspect 1, wherein the sleeve and the plurality of cell culture substrate layers are removably disposed within the bioreactor vessel.

Aspect 3 pertains to the fixed bed bioreactor assembly of Aspect 2, wherein the sleeve and the plurality of cell culture substrate layers can slide together at least partially out of the bioreactor vessel through an opening in a top or a bottom of the bioreactor vessel.

Aspect 4 pertains to the fixed bed bioreactor assembly of Aspect 3, wherein, when slide at least partially out of the bioreactor vessel, the at least one sample access window is clear of the sidewall of the bioreactor vessel such that the one or more layers of the cell culture substrate can be removed for sampling.

Aspect 5 pertains to the fixed bed bioreactor assembly of any of Aspects 1-4, wherein the sleeve wraps circumferentially around the plurality of cell culture substrate layers.

Aspect 6 pertains to the fixed bed bioreactor assembly of Aspect 5, wherein the opening of the sample access window is defined by a portion of the sleeve that wraps around less of the circumference of the plurality of cell culture substrate layers than another portion of the sleeve.

Aspect 7 pertains to the fixed bed bioreactor assembly of any of Aspects 1-6, wherein the opening has a width that is less than a maximum width of the one or more layers of the cell culture substrate to be removed.

Aspect 8 pertains to the fixed bed bioreactor assembly of any of Aspects 1-7, wherein the at least one sample access window is disposed at multiple predetermined locations along the height of the bioreactor vessel.

Aspect 9 pertains to the fixed bed bioreactor assembly of Aspect 8, wherein the at least one sample access window is disposed in one or more of a bottom region of the bioreactor vessel, a middle region of the bioreactor vessel, and a top region of the bioreactor vessel.

Aspect 10 pertains to the fixed bed bioreactor assembly of any of Aspects 1-9, further comprising one or more seals disposed on the sleeve.

Aspect 11 pertains to the fixed bed bioreactor assembly of Aspect 10, wherein the seals are disposed along the edges of the opening of the sample access window.

Aspect 12 pertains to the fixed bed bioreactor assembly of Aspect 10 or Aspect 11, wherein the seals form an interference fit with the sidewall of the bioreactor vessel.

Aspect 13 pertains to the fixed bed bioreactor assembly of any of Aspects 1-12, wherein each layer of the plurality of cell culture substrate layers comprises a plurality of woven fibers.

Aspect 14 pertains to the fixed bed bioreactor assembly of any of Aspects 1-13, wherein the cell culture substrate comprises a plurality of layers arranged in a stacked configuration.

Aspect 15 pertains to a method of sampling a cell culture substrate from a bioreactor vessel, the method comprising: removing a sleeve of cell culture substrate layers stacked within the sleeve from a bioreactor vessel through a top or bottom of a bioreactor vessel; removing one or more layers of the cell culture substrate through a sample access window disposed in a side of the sleeve, the sample access window being an opening formed in the sleeve and exposed at least a portion of the cell culture substrate layers.

Aspect 16 pertains to the method of Aspect 15, further comprising, after removing the one or more layers, reinserting the sleeve of cell culture substrate layers into the bioreactor vessel.

Aspect 17 pertains to the method of Aspect 15 or Aspect 16, wherein the removing of the one or more layers occurs during an active cell culture.

Aspect 18 pertains to a fixed bed bioreactor system for culturing cells comprising: a bioreactor vessel comprising an interior space for culturing cells and a sidewall at least partly defining the interior space; at least one port in the sidewall of the bioreactor vessel; a cell culture substrate disposed in the interior space; and a sampling reel disposed on an exterior of the bioreactor vessel, the sampling reel being configured to rotate about its central axis to aseptically extract a sample substrate from interior space.

Aspect 19 pertains to the fixed bed bioreactor system of Aspect 18, wherein the sampling reel comprises a passthrough opening through which the substrate sample is able to pass from the interior space and into the sampling reel.

Aspect 20 pertains to the fixed bed bioreactor system of Aspect 18 or Aspect 19, wherein the sampling reel is affixed to the exterior of the bioreactor vessel wall via a saddle housing forming at least a portion of the outer shell of the sampling reel.

Aspect 21 pertains to the fixed bed bioreactor system of Aspect 19 or Aspect 20, wherein the passthrough opening of the sampling reel is aligned with the at least one port.

Aspect 22 pertains to the fixed bed bioreactor system of any of Aspects 18-21, wherein the sampling reel further comprises a housing cap forming at least a portion of the outer shell of the sampling reel.

Aspect 23 pertains to the fixed bed bioreactor system of any of Aspects 18-22, wherein the sampling reel further comprises a sample opening.

Aspect 24 pertains to the fixed bed bioreactor system of Aspect 23, wherein the sample opening is disposed between the housing cap and saddle housing.

Aspect 25 pertains to the fixed bed bioreactor system of Aspect 23 or Aspect 24, wherein the sample opening is configured to allow a substrate sample to be extracted from the sampling reel via the sample opening to an exterior of the bioreactor system.

Aspect 26 pertains to the fixed bed bioreactor system of any of Aspects 18-25, wherein the sampling reel further comprises a spool configured to grip a portion of the sample substrate, the spool being rotatable such that when the spool rotates, the sample substrate is wound around the spool.

Aspect 27 pertains to the fixed bed bioreactor system of Aspect 26, wherein the sampling reel further comprises a sample drum disposed between the spool and the outer shell of the sampling reel.

Aspect 28 pertains to the fixed bed bioreactor system of Aspect 27, wherein the sampling reel further comprises series of stationary o-rings affixed to the saddle housing, and configured to create seals in a space between the saddle housing and the sample drum, to enable the aseptic sampling of the sample substrate.

Aspect 29 pertains to the fixed bed bioreactor system of Aspect 27 or Aspect 28, the sample drum further comprises a drum opening configured to allow the sample substrate to pass within the sample drum to the spool.

Aspect 30 pertains to the fixed bed bioreactor system of any of Aspects 18-29, wherein the sampling reel is configured to rotate in a first direction to extract the sample substrate from the interior space, and to rotate in a second direction opposite to the first direction to release the sample substrate from the sampling reel to an exterior of the bioreactor system.

Aspect 31 pertains to the fixed bed bioreactor system of any of Aspects 18-30, wherein the substrate sample lifted through an opening of the sampling reel to an exterior of the bioreactor system via a curved or wedged surface on an edge of the passthrough opening.

Aspect 32 pertains to the fixed bed bioreactor system of any of Aspects 18-31, wherein the cell culture substrate comprises a plurality of woven fibers.

Aspect 33 pertains to the fixed bed bioreactor system of any of Aspects 18-32, wherein the cell culture substrate comprises a plurality of layers arranged in a stacked configuration.

Aspect 34 pertains to the fixed bed bioreactor system of any of Aspects 18-33, wherein the bioreactor vessel is configured for aseptic removal of the sample substrate from the interior space.

Aspect 35 pertains to a fixed bed bioreactor system for culturing cells comprising: a bioreactor vessel comprising an interior space for culturing cells and a sidewall at least partly defining the interior space; at least one port in the sidewall of the bioreactor vessel; and a cell culture substrate disposed in the interior space and having a structurally defined surface for culturing cells thereon, the structurally defined surface defining an ordered and regular array of openings through a thickness of the cell culture substrate, wherein at least a portion of the cell culture substrate comprises a sample substrate, the sample substrate being defined by a separation boundary between the sample substrate and a remainder of the cell culture substrate, wherein the separation boundary is configured to separate the sample substrate from the remainder of the cell culture substrate, and wherein the at least one port is sized so that the sample substrate can be removed from the interior space through the at least one port.

Aspect 36 pertains to the fixed bed bioreactor system of Aspect 35, wherein the separation boundary comprises at least one of the following: perforations in, cuts into or through, or locally thinned portions of the cell culture substrate.

Aspect 37 pertains to the fixed bed bioreactor system of Aspect 35 or Aspect 36, wherein the separation boundary further comprises an attachment material between the sample substrate and the remainder of the substrate, the attachment material being configured to release from one or both of the sample substrate and the remainder of the substrate under tension.

Aspect 38 pertains to the fixed bed bioreactor system of any of Aspects 35-37, further comprising a plurality of sample substrates.

Aspect 39 pertains to the fixed bed bioreactor system of Aspect 38, wherein at least two of the plurality of sample substrates are separated from each other by a portion of the remainder of the cell culture substrate that is not one of the plurality of sample substrates.

Aspect 40 pertains to the fixed bed bioreactor system of Aspect 38 or Aspect 39, wherein at least a portion of the plurality of sample substrates are separated from each other by the separation boundary and without any of the remainder of the cell culture substrate that is not one of the plurality of sample substrate therebetween.

Aspect 41 pertains to the fixed bed bioreactor system of any of Aspects 35-40, wherein the cell culture substrate comprises a circular disk shape.

Aspect 42 pertains to the fixed bed bioreactor system of any of Aspects 35-41, wherein the sample substrate comprises at least one of the following shapes: square, rectangular, pie-shaped, or tapered.

Aspect 43 pertains to the fixed bed bioreactor system of Aspect 42, wherein the sample substrate is tapered with the narrow tapered end on a periphery of the cell culture substrate.

Aspect 44 pertains to the fixed bed bioreactor system of Aspect 42 or Aspect 43, wherein the sample substrate is square or rectangular on a first end of the sample substrate within the periphery of the cell culture substrate, and is tapered on a second end at the periphery of the cell culture substrate.

Aspect 45 pertains to the fixed bed bioreactor system of Aspect 42, wherein the cell culture substrate is circular and comprises a plurality of pie-shaped sample substrates.

Aspect 46 pertains to the fixed bed bioreactor system of any of Aspects 35-45, wherein the structurally defined surface comprises one of more fibers.

Aspect 47 pertains to the fixed bed bioreactor system of Aspect 46, wherein the cell culture substrate comprises a plurality of woven fibers.

Aspect 48 pertains to the fixed bed bioreactor system of any of Aspects 35-47, wherein the cell culture substrate comprises a plurality of layers are arranged in a stacked configuration.

Aspect 49 pertains to the fixed bed bioreactor system of any of Aspects 35-48, wherein the bioreactor vessel is configured for aseptic removal of the sample substrate from the interior space.

Aspect 50 pertains to the fixed bed bioreactor system of any of Aspects 35-49, further comprising a tether comprising a first end that is attached to the sample substrate, the tether being configured to pull the sample substrate out of the interior space through the port.

Aspect 51 pertains to the fixed bed bioreactor system of any of Aspects 35-50, further comprising a capture device disposed outside of the interior space and configured to contain the sample substrate after the sample substrate is removed from the interior space via the at least one port.

Aspect 52 pertains to the fixed bed bioreactor system of Aspect 51, wherein the capture device comprises flexible walls, pleated walls, or folds that enable the capture device to have a variable interior volume.

Aspect 53 pertains to the fixed bed bioreactor system of any of Aspects 50-52, further comprising a plug disposed along the tether between the first end and the second end of the tether, wherein the plug is configured to form an aseptic seal in the port while the sample substrate remains in the interior space.

Aspect 54 pertains to the fixed bed bioreactor system of Aspect 53, wherein the plug is configured to dislodge from the port when the second end of the tether is pulled away from the port with a predetermined force.

Aspect 55 pertains to the fixed bed bioreactor system of any of Aspects 51-54, wherein the capture device is attached to the port at least while the sample substrate remains in the interior space.

Aspect 56 pertains to the fixed bed bioreactor system of Aspect 55, wherein the capture device is removably attached to the port.

Aspect 57 pertains to the fixed bed bioreactor system of any of Aspects 51-54, wherein the capture device is separate from the port and the bioreactor vessel, the capture device being configured to temporarily attach to the port during removal of the sample substrate from the interior space via the port.

1-34. (canceled)

35. A fixed bed bioreactor system for culturing cells comprising:
a bioreactor vessel comprising an interior space for culturing cells and a sidewall at least partly defining the interior space:
at least one port in the sidewall of the bioreactor vessel; and
a cell culture substrate disposed in the interior space and having a structurally defined surface for culturing cells thereon, the structurally defined surface defining an ordered and regular array of openings through a thickness of the cell culture substrate,
wherein at least a portion of the cell culture substrate comprises a sample substrate, the sample substrate being defined by a separation boundary between the sample substrate and a remainder of the cell culture substrate,
wherein the separation boundary is configured to separate the sample substrate from the remainder of the cell culture substrate, and
wherein the at least one port is sized so that the sample substrate can be removed from the interior space through the at least one port.

36. The fixed bed bioreactor system of claim 35, wherein the separation boundary comprises at least one of the following: perforations in, cuts into or through, or locally thinned portions of the cell culture substrate.

37. The fixed bed bioreactor system of claim 35, wherein the separation boundary further comprises an attachment material between the sample substrate and the remainder of the substrate, the attachment material being configured to release from one or both of the sample substrate and the remainder of the substrate under tension.

38. The fixed bed bioreactor system of claim 35, further comprising a plurality of sample substrates.

39. The fixed bed bioreactor system of claim 38, wherein at least two of the plurality of sample substrates are separated from each other by a portion of the remainder of the cell culture substrate that is not one of the plurality of sample substrates.

40. The fixed bed bioreactor system of claim 38, wherein at least a portion of the plurality of sample substrates are separated from each other by the separation boundary and without any of the remainder of the cell culture substrate that is not one of the plurality of sample substrate therebetween.

41. The fixed bed bioreactor system of claim 35, wherein the cell culture substrate comprises a circular disk shape.

42. The fixed bed bioreactor system of claim 35, wherein the sample substrate comprises at least one of the following shapes: square, rectangular, pie-shaped, or tapered.

43. The fixed bed bioreactor system of claim 42, wherein the sample substrate is tapered with the narrow tapered end on a periphery of the cell culture substrate.

44. The fixed bed bioreactor system of claim 42, wherein the sample substrate is square or rectangular on a first end of the sample substrate within the periphery of the cell culture substrate, and is tapered on a second end at the periphery of the cell culture substrate.

45. The fixed bed bioreactor system of claim 42, wherein the cell culture substrate is circular and comprises a plurality of pie-shaped sample substrates.

46. The fixed bed bioreactor system of claim 35, wherein the structurally defined surface comprises one of more fibers.

47. The fixed bed bioreactor system of claim 46, wherein the cell culture substrate comprises a plurality of woven fibers.

48. The fixed bed bioreactor system of claim 35, wherein the cell culture substrate comprises a plurality of layers are arranged in a stacked configuration.

49. The fixed bed bioreactor system of claim 35, wherein the bioreactor vessel is configured for aseptic removal of the sample substrate from the interior space.

50. The fixed bed bioreactor system of claim 35, further comprising a tether comprising a first end that is attached to the sample substrate, the tether being configured to pull the sample substrate out of the interior space through the port.

51. The fixed bed bioreactor system of claim 35, further comprising a capture device disposed outside of the interior space and configured to contain the sample substrate after the sample substrate is removed from the interior space via the at least one port.

52. The fixed bed bioreactor system of claim 51, wherein the capture device comprises flexible walls, pleated walls, or folds that enable the capture device to have a variable interior volume.

53. The fixed bed bioreactor system of claim 50, further comprising a plug disposed along the tether between the first end and the second end of the tether, wherein the plug is configured to form an aseptic seal in the port while the sample substrate remains in the interior space.

54. The fixed bed bioreactor system of claim 53, wherein the plug is configured to dislodge from the port when the second end of the tether is pulled away from the port with a predetermined force.

55. (canceled)

56. (canceled)

57. (canceled)
